(19) United States
(12) Patent Application Publication
(10) Pub. No.: US 2018/0273904 A1
(43) Pub. Date: Sep. 27, 2018

(54) SPONTANEOUSLY BEATING CARDIAC ORGANOID CONSTRUCTS AND INTEGRATED BODY-ON-CHIP APPARATUS CONTAINING THE SAME

(71) Applicant: Wake Forest University Health Sciences, Winston-Salem, NC (US)

(72) Inventor: Aleksander Skardal, Clrmmons, NC (US)

(21) Appl. No.: 15/765,077

(22) PCT Filed: Sep. 30, 2016
(86) PCT No.: PCT/US2016/054607

§ 371 Date: Mar. 30, 2018

Related U.S. Application Data
(60) Provisional application No. 62/236,348, filed on Oct. 2, 2015.

(51) Int. Cl. C12N 5/077; C12M 3/00; C12M 1/34; C12N 5/071; C12M 1/00

(52) U.S. Cl. CPC C12N 5/0657; C12M 21/08; C12M 41/46; C12N 5/0671; C12M 29/00; C12N 2502/1329; C12N 2533/54; C12N 2513/00; C12N 2501/999; C12N 2502/14; C12N 2533/56

(57) ABSTRACT

A method of making a cardiac construct is carried out by depositing a mixture comprising live mammalian cardiac cells (e.g., individual cells, organoids, or spheroids), fibrinogen, gelatin, and water on a support to form an intermediate cardiac construct; optionally co-depositing a structural support material (e.g., polycaprolactone) with the mixture in a configuration that supports the intermediate construct; and then contacting thrombin to the construct in an amount effective to cross-link the fibrinogen and produce a cardiac construct comprised of live cardiac cells that together spontaneously beat in a fibrin hydrogel. Constructs made and methods of using the same are also described.

### RELATED APPLICATIONS

This application claims the benefit of and priority to U.S. Provisional Application Ser. No. 62/236,348, filed Oct. 2, 2015, the disclosure of which is hereby incorporated by reference herein in its entirety.

### GOVERNMENT SUPPORT

This invention was made with government support under Contract No. N66001-13-C-2027 awarded by the Defense Threat Reduction Agency (DTRA) under Space and Naval Warfare Systems Center Pacific (SSC PACIFIC), and Grant No. NCI CCSG P30CA012197 awarded by the National Cancer Institute. The US Government has certain rights to this invention.

### FIELD OF THE INVENTION

The present invention concerns organoids useful for in vitro physiology and pharmacology investigations, and integrated systems containing the same.

### BACKGROUND OF THE INVENTION

There is a critical need for improved biological model systems for testing the effects of drugs and chemical and biological agents on the body.^{1,2} Currently, animal models serve as the gold standard for testing, but the drawbacks associated with such models include high costs and uncertainties in interpretation of the results, as responses to external stimuli in animals are not necessarily predictive of those in humans.³ Due to interspecies differences and variability of the results, animal models are often poor predictors of human efficacy and toxicology, contributing to drug attrition rates.⁴ In vitro systems using human tissues would help circumvent this issue; however, traditional in vitro 2D cultures fail to recapitulate the 3D microenvironment of in vivo tissues.^{5,6} Drug diffusion kinetics vary dramatically, drug doses effective in 2D are often ineffective when scaled to patients, and cell-cell/cell-extracellular matrix (ECM) interactions in 2D are often inaccurate, contributing to loss or change of cell function.^{5,7,8} Bioengineered tissue construct platforms have evolved, which can better mimic the structure and cellular heterogeneity of in vivo tissue, and are also suitable for in vitro screening applications. These technologies have the potential to recapitulate the dynamic role of cell-cell, cell-ECM, and mechanical interactions of in vivo tissues. Furthermore, incorporation of supportive cells, such as endothelial cells and fibroblasts, and physical matrix components, can more completely mimic the native tissue microenvironment.

For in vitro systems to serve as tools capable of reflecting human biology, key physiological features and toxicology endpoints need to be included in their design to allow for informative and reliable efficacy, pharmacokinetics, and toxicity testing. A “body-on-a-chip” device that can simulate multi-tissue interactions under physiological fluid flow conditions holds the potential to meet these requirements. A microfluidic chip system, designed to mimic responses found in a human, should be capable of producing rapid, reliable predictions of elicited reactions of the body to drugs, biologicals, and chemicals. This system would also have the potential to advance the development of new technologies for streamlining the drug development pipeline. Continued advancement in microengineering and microfluidics technologies have further contributed to the evolution of 3D human tissue-on-a chip models and their more widespread implementation.⁹ A variety of microscale models of human organs-on-chips as well as disease models currently exist, including liver, spleen, lung, marrow, muscle, and cardiac tissues.¹⁰

Usually, implementation of highly functioning cells, such as primary adult hepatocytes¹¹ and adult or induced pluripotent stem cell-derived cardiomyocytes^{12,13} for drug discovery applications has been a technically difficult and expensive process.¹⁴ As previously mentioned, traditional tissue culture conditions are typically not sufficient for long-term culture and maintenance of physiological function, especially for the culture of primary hepatocytes. Tissue culture dishes have three major differences from the tissue where the cells were isolated: surface topography, surface stiffness, and most importantly, a 2D rather than 3D architecture. As a consequence, 2D culture places a selective pressure on cells, substantially altering their original molecular and phenotypic properties. Fortunately, 3D biofabrication approaches that leverage biomaterials¹⁵ and techniques such as bioprinting⁶⁻¹⁸ allow for creation of tissue constructs complete with accurate architecture, physiology, and tissue-specific signals, thereby forming physiologically-mimicking environments that effectively increase in vitro tissue function. The ability to replicate in vivo tissue functionality in vitro enables development of cost-effective high-throughput platforms to rapidly screen or test drugs, drug candidates, and chemical agents with minimal reliance on time consuming and expensive in vivo experiments conducted in animal models. If mass produced, such organ-on-a-chip systems could be an asset to the pharmaceutical industry for drug candidate screening, and to scientists investigating a variety of diseases.¹⁹

### SUMMARY OF THE INVENTION

We had originally developed the tissue-mimicking bioink system described in A. Skardal et al., A hydrogel bioink toolkit for mimicking native tissue biochemical and mechanical properties in bioprinted tissue constructs, Acta Biomater 25:24-34 (Epub 22 Jul. 2015) (see also U.S. Provisional Application No. 62/068,218; Filed Oct. 24, 2014) to provide a platform that could be used with any, or most, tissue types. We believe that this is still the case, based on our use of components of this system with a wide variety of cell types from various tissues and organs.

However, when we transitioned to cardiac organoids, we found that—when incorporating these cardiac organoids into a hydrogel or of the type described in the works above, with or without the cardiac-specific extracellular matrix components—the organoids, which normally demonstrated spontaneous beating (or pulsing) behavior, would stop beating upon enapsulation. We suspected that this might be due to the rigidity of the covalent crosslinks within the hydrogel bioink. To be clear, the bioink gels are still relatively soft to the human touch, but we thought that from the perspective of the cardiac cells in the construct, the surrounding hyaluronic acid, gelatin matrix, polyethylene glycol-based crosslinker matrix, may have been difficult to either interact with, or didn't “give” as easily, preventing the cardiac organoids to beat. Alternatively, there may have been some chemical component that prevented the beating through signaling. The fibrin-based hydrogel materials described herein have been found to overcome this problem.

Accordingly, a first aspect of the invention is a method of making a cardiac construct, comprising: depositing a mixture comprising live mammalian cardiac cells (e.g., individual cells, organoids, or spheroids), fibrinogen, gelatin, and water on a support to form an intermediate cardiac construct; optionally co-depositing a structural support material (e.g., polycaprolactone) with the mixture in a configuration that supports the intermediate construct; and the contacting thrombin to the construct in an amount effective to cross-link the fibrinogen and produce (with intervening incubation as necessary, depending on the maturity of the cardiac cells to begin with) a cardiac construct comprised of live cardiac cells that together spontaneously beat in a fibrin hydrogel.

A further aspect of the invention is an apparatus, comprising:

(a) a first chamber having an inlet and an outlet; and

(b) a cardiac construct in the primary chamber, the cardiac construct comprising a cross-linked fibrin hydrogel, and cardiac cells that spontaneously beat together in the hydrogel.

In some embodiments, the apparatus further includes:

(d) at least one secondary chamber in fluid communication with the primary chamber; and

(e) a live mammalian liver tissue construct in the secondary chamber.

In some embodiments, the apparatus further includes:

(f) at least one additional secondary chamber in fluid communication with the primary and/or secondary chambers (e.g. through a conduit therebetween); and

(g) at least one additional live tissue construct (e.g. lung, blood vessel, intestine, brain, colon, etc.) independently selected in each additional secondary chamber.

Additional aspects and embodiments of the present invention are explained in greater detail in the specification and Figures set forth below. The disclosures of all United States Patent references cited herein are to be incorporated by reference herein in their entirety.

### BRIEF DESCRIPTION OF THE DRAWINGS

FIG. 1. Liver organoids retain dramatically increased baseline liver function and metabolism compared to 2D hepatocyte cultures, and respond to toxins. a-b) Normalized a) albumin and b) urea secretion into media, analyzed by ELISA and colorimetric assays show dramatically increased functional output in the 3D organoid format in comparison to 2D hepatocyte sandwich cultures. Quantification of the diazepam metabolites c) temazepam, d) noridazepam, and e) oxazepam primarily by CYP2C19 and CYP3A4. The toxic effects of liver organoid treatment with the drug troglitazone depicted by f) a dose response analysis assessed by ATP quantification, and g) phospholipid accumulation in a subset (0 μM, 25 μM, 50 μM, and 100 μM) of troglitazone doses. Statistical significance: *p<0.05 between 3D and 2D comparisons at each time point. Scale bars—300 μm.

FIG. 2. Organoid construct bioprinting and on-chip integration. a-c) Organoid construct bioprinting using hydrogel bioink and spheroid organoid building blocks is printed within PCL support structures on modular chips for integration into the fluidic system. a) The bioprinter used for bioprinting, developed in-house. b) A depiction of the bioprinted construct geometry using organoid specific hydrogel bioinks. Bioprinted c) liver and d) cardiac organoid constructs. e) A depiction of integrating organoid constructs into the microfluidic microreactor system. Bioprinted liver constructs on 7 mm×7 mm coverslips are transferred into the central chamber of the PDMS microreactor devices. Devices are sealed, fluid connections are completed and flow is initiated at 10 L/min, drawing media from an in-line media reservoir.

FIG. 3. On-chip liver organoid viability and functional response to acetaminophen and an N-acetyl-L-cysteine countermeasure. a-c) Long term viability of bioprinted liver constructs. LIVE/DEAD stained images depict relatively consistent cell viability over 4 weeks. Green—Calcein AM-stained viable cells; Red—Ethidium homodimer-stained dead cells. d-g) Liver organoids respond to acetaminophen toxicity and are rescued by NAC. Viability as determined by LIVE/DEAD staining on day 14. Organoids were exposed to d) a 0 mM APAP control, e) 1 mM APAP, f) 10 mM APAP, or g) 10 mM APAP with 20 mM N-acetyl-L-cysteine. Scale bar—100 μm. h-k) Analysis of media aliquots suggest APAP induces loss of function and cell death, while NAC has the capability to mitigate these negative effects. Quantification of h) human albumin, i) urea, j) lactate dehydrogenase, and k) alpha-GST. Albumin and urea output are negatively effected by APAP treatments, while NAC decreases this reduction in secretion. LDH and alpha-GST are low in control and APAP+NAC groups suggesting viable cells, while APAP induces elevated or spiked levels, indicating apoptosis and release of LDH and alpha-GST into the media. Statistical significance: *p<0.05 between Control and APAP; #p<0.05 APAP+NAC and APAP.

FIG. 4. Monitoring of cardiac organoid beating and modulation of beating rate as an effect of drug treatment. a) A depiction and images of the on-chip camera system used to capture real-time video of beating cardiac organoids during culture within the ECHO platform. b) Screen capture from a video of a beating cardiac organoid within the microfluidic system, and c) screen capture of a thresholded pixel movement binarization of the beating cardiac organoid, generated by custom written MatLab code, allowing quantification of beat rates. d) Beating output plot under baseline conditions from which beating rate is determined. e-g) Cardiac organoid beat peak plots altered from baseline using e) isoproterenol, or f) quinidine. G-h) Cardiac organoid response to epinephrine and propranolol. g) Cardiac organoids experience a dose dependent increase in beating rate ranging from 1 to almost 2-fold with increasing epinephrine concentration before reaching a beating rate plateau with 5 μM epinephrine and higher. h) Initial incubation with propranolol concentrations ranging from 0 to 20 μM results in a dose dependent decrease in beating rate after administration of 5 μM epinephrine.

FIG. 5. Combining liver and cardiac modules results in a biological system capable of an integrated response to drugs. a) A schematic depicting the integrated liver and cardiac system for testing dual-organoid response to environmental manipulations. b) Incorporation of liver organoids results in variation in cardiac organoid response to both 0.1 μM propranolol and 0.5 μM epinephrine. c) The effects of liver metabolic activity on downstream cardiac beating rates. BPM values increase from baseline with 0.5 μM epinephrine; increased rates from epinephrine are blocked by 0.1 μM propranolol. When liver organoids are present and permitted to metabolize 0.1 μM propranolol, 0.1 μM epinephrine is capable of inducing an increased BPM value. Statistical significance: *<0.05. d-g) Cardiac organoid beat peak plots corresponding to the values presented in panel c).

FIG. 6. Sensor integration in the multi-organoid ECHO body-on-a-chip platform. a) An overview photograph illustrating the components of an assembled ECHO system. b) Incorporation of the bubble trap module reduces turbulence, resulting in consistent and smooth flow over time. c) A temperature probe monitors the environmental temperature of the fluid flowing through the ECHO fluidics and responds to environmental changes, illustrated by a drop in temperature upon opening of the incubator. d) An optics based pH sensor i) operates using a light emitting diode, filter, and photodiode to measure media color; ii) output sensitivity demonstrated using 0.5 pH decreases and increases in the system. e) An oxygen sensor measures O2 levels using an LED and on board camera and photodiode system. f) A schematic depicting the microfluidic multiplexed albumin, α-GST, and CK-MB electrochemical detection module. g) Impedance readings for the albumin electrochemical sensor under bare electrode, self-assembled monolayer, CK aptamer, media, 1 ng/mL CK, 10 ng/mL CK, and 100 ng/mL conditions. h) Measurement of albumin, α-GST, and CK-MB over a 12-hour integrated liver and cardiac ECHO system time-course.

### DETAILED DESCRIPTION OF ILLUSTRATIVE EMBODIMENTS

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

### A. Definitions

“Cells” used in the present invention are, in general, animal cells, particularly mammalian and primate cells, examples of which include but are not limited to human, dog, cat, rabbit, monkey, chimpanzee, cow, pig, goat. The cells are preferably differentiated at least in part to a particular cell or tissue type, such as liver, intestine, pancreas, lymph node, smooth muscle, skeletal muscle, central nerve, peripheral nerve, skin, immune system, etc. Some cells may be cancer cells, as discussed further below, in which case they optionally but preferably express (naturally, or by recombinant techniques) a detectable compound, as also discussed further below.

“Three dimensional tissue construct” as used herein, and refers to a composition of live cells, typically in a carrier media, arranged in a three-dimensional or multi-layered configuration (as opposed to a monolayer). Suitable carrier media include hydrogels, such as cross-linked hydrogels as described below. Such constructs may comprise one differentiated cell type, or two or more differentiated cell types, depending upon the particular tissue or organ being modeled or emulated. Some organoids may comprise cancer cells, as discussed further below. Where the constructs comprise cancer cells, they may include tissue cells, and/or may include a tissue mimic without cells, such as an extracellular matrix (or proteins or polymers derived therefrom), hyaluronic acid, gelatin, collagen, alginate, etc., including combinations thereof. Thus in some embodiments, cells are mixed together with the extracellular matrix, or cross-linked matrix, to form the construct, while in other embodiments cell aggregates such as spheroids or organoids may be pre-formed and then combined with the extracellular matrix.

“Growth media” as used herein may be any natural or artificial growth media (typically an aqueous liquid) that sustains the cells used in carrying out the present invention. Examples include, but are not limited to, an essential media or minimal essential media (MEM), or variations thereof such as Eagle's minimal essential medium (EMEM) and Dulbecco's modified Eagle medium (DMEM), as well as blood, blood serum, blood plasma, lymph fluid, etc., including synthetic mimics thereof. In some embodiments, the growth media includes a pH color indicator (e.g., phenol red).

“Test compound” or “candidate compound” as used herein may be any compound for which a pharmacological or physiological activity, on cardiac tissue and/or other tissue, or an interaction between two test compounds, is to be determined. For demonstrative purposes, isoproterenol and quinidine are used separately below as test compounds to examine them independently, while propranolol and epinephrine are administered concurrently or in combination with one another as test compounds to examine the interaction therebetween. However, any compound may be used, typically organic compounds such as proteins, peptides, nucleic acids, and small organic compounds (aliphatic, aromatic, and mixed aliphatic/aromatic compounds) may be used. Candidate compounds may be generated by any suitable techniques, including randomly generated by combinatorial techniques, and/or rationally designed based on particular targets. Where a drug interaction is to be studied, two (or more) test compounds may be administered concurrently, and one (or both) may be known compounds, for which the possible combined effect is to be determined.

### B. Compositions for Making Tissue Constructs in General

Compositions of the present invention may comprise live cells in a “bioink,” where the “bioink” is in turn comprised of a cross-linkable polymer, a post-deposition crosslinking group or agent; and other optional ingredients, including but not limited to growth factors, initiators (e.g., of cross-linking), water (to balance), etc. The compositions are preferably in the form of a hydrogel. Various components and properties of the compositions are discussed further below.

Cells.

As noted above, cells used to carry out the present invention are preferably animal cells (e.g., bird, reptile, amphibian, etc.) and in some embodiments are preferably mammalian cells (e.g., dog, cat, mouse, rat, monkey, ape, human). The cells may be differentiated or undifferentiated cells, but are in some embodiments tissue cells (e.g., liver cells such as hepatocytes, pancreatic cells, cardiac muscle cells, skeletal muscle cells, etc.).

Choice of cells will depend upon the particular organoid being created. For example, for a liver organoid, liver hepatocyte cells may be used. For a peripheral or central nerve organoid, peripheral nerve cells, central nerve cells, glia cells, or combinations thereof may be used. For a bone organoid, bone osteoblast cells, bone osteoclast cells, or combinations thereof may be used. For a lung organoid, lung airway epithelial cells may be used. For a lymph node organoid, follicular dendritic lymph cells, fibroblastic reticular lymph cells, leukocytes, B cells, T cells, or combinations thereof may be used. For a smooth or skeletal muscle organoid, smooth muscle cells, skeletal muscle cells, or combinations thereof may be used. For a skin organoid, skin keratinocytes, skin melanocytes, or combinations thereof may be used. The cells may be differentiated upon initial incorporation into the composition, or undifferentiated cells that are subsequently differentiated may be used. Additional cells may be added to any of the compositions described above, and cancer cells as described below may be added to primary or “first” organoids, as described below.

Cancer cells optionally used in the present invention may be any type of cancer cell, including but not limited to melanoma, carcinoma, sarcoma, blastoma, glioma, and astrocytoma cells, etc.

The cells may be incorporated into the composition in any suitable form, including as unencapsulated cells, or as cells previously encapsulated in spheroids, or pre-formed organoids (as noted above). Animal tissue cells encapsulated or contained in polymer spheroids can be produced in accordance with known techniques, or in some cases are commercially available (see, e.g., Insphero AG, 3D Hepg2 Liver Microtissue Spheroids (2012); Inspherio AG, 3D InSight™ Human Liver Microtissues, (2012)).

Cross-Linkable Prepolymers.

Any suitable prepolymer can be used to carry out the present invention, so long as it can be further cross-linked to increase the elastic modulus thereof after deposition when employed in the methods described herein.

In some embodiments, the prepolymer is formed from the at least partial crosslinking reaction of: (i) an oligosaccharide (e.g., hyaluronic acid, collagen, combinations thereof and particularly thiol-substituted derivatives thereof) and (ii) a first crosslinking agent (e.g., a thiol-reactive crosslinking agent, such as polyalkylene glycol diacrylate, polyalkylene glycol methacrylate, etc., and particularly polyethylene glycol diacrylate, etc.; thiolated crosslinking agent to create thiol-thiol disulfide bonds; gold nanoparticles gold functionalized crosslinkers forming thiol-gold bonds; etc., including combinations thereof).

Cross-Linking Group.

In some embodiments, the compositions include a post-deposition crosslinking group. Any suitable crosslinking groups can be used, including but not limited to multi-arm thiol-reactive crosslinking agent, such as polyethylene glycol dialkyne, other alkyne-functionalized groups, acrylate or methacrylate groups, etc., including combinations thereof.

Initiators.

Compositions of the invention may optionally, but in some embodiments preferably, include an initiator (e.g., a thermal or photoinitiator). Any suitable initiator that catalyzes the reaction between said prepolymer and the second (or post-deposition) crosslinking group (e.g., upon heating or upon exposure to light), may be employed.

Growth Factors.

Compositions of the invention may optionally, but in some embodiments preferably, include at least one growth factor (e.g., appropriate for the particular cells included, and/or for the particular tissue substitute being produced). In some embodiments, growth factors and/or other growth promoting proteins may be provided in a decellularized extracellular matrix composition (“ECM”) from a tissue corresponding to the tissue cells (e.g., decellularized extracellular liver matrix when the live animal cells are liver cells; decellularized extracellular cardiac muscle matrix when the live animal cells are cardiac muscle cells; decellularized skeletal muscle matrix when the live animal cells are skeletal muscle cells; etc.). Additional collagens, glycosaminoglycans, and/or elastin (e.g., which may be added to supplement the extracellular matrix composition), etc., may also be included.

Elastic Modulus.

The composition preferably has an elastic modulus, at room temperature and atmospheric pressure, sufficiently low such that it can be manipulated and deposited on a substrate by whatever deposition method is employed (e.g., extrusion deposition). Further, the composition optionally, but in some embodiments preferably, has an elastic modulus, again at room temperature and atmospheric pressure, sufficiently high so that it will substantially retain the shape or configuration in which it is deposited until subsequent cross-linking (whether that cross-linking be spontaneous, thermal or photo-initiated, etc.). In some embodiments, the composition, prior to deposition, has a stiffness of from 0.05, 0.1 or 0.5 to 1, 5 or 10 kiloPascals, or more, at room temperature and atmospheric pressure.

### C. Methods and Compositions for Making Cardiac Constructs in Particular

As noted above, the present invention provides a method of making a cardiac construct, comprising: depositing a mixture comprising live mammalian cardiac cells (e.g., individual cells, organoids, or spheroids), fibrinogen, gelatin, and water on a support to form an intermediate cardiac construct; optionally co-depositing a structural support material (e.g., polycaprolactone) with the mixture in a configuration that supports the intermediate construct; and then contacting thrombin to the construct in an amount effective to cross-link the fibrinogen and produce (with intervening incubation as necessary, depending on the maturity of the cardiac cells to begin with) a cardiac construct comprised of live cardiac cells that together spontaneously beat in a fibrin hydrogel.

In some embodiments, the cardiac cells are in the form of organoids produced by hanging-drop culture of cardiomyocytes. See, e.g., US 2011/0287470 to Stoppini.

In some embodiments, the cardiac construct (specifically, the cardiac cells therein) exhibits spontaneous beating that is increased in frequency by the administration of isoproterenol in an effective amount and decreased in frequency by the administration of quinidine in an effective amount.

In some embodiments, the cardiac construct (specifically, the cardiac cells therein) express VEGF, actinin, and/or cardiac troponin-T.

As with the general bioink described in the section above, unmodified gelatin can be added to the fibrinogen in order to thicken it into an extrudable material that can be bioprinted using bioprinting devices. As this gelatin is not crosslinked, upon incubation at physiological temperature (37 degrees C.) after bioprinting a cardiac construct, the gelatin eventually dissolves and leaches out of the construct, leaving behind only the crosslinked fibrin and the beating cardiac construct.

### D. Methods of Making Devices

In one non-limiting, but preferred, method of use, the compositions are used in a method of making each particular construct in a device as described herein. Such a method generally comprises the steps of:

(a) providing a reservoir containing an extrudable hydrogel composition as described above, then

(b) depositing the hydrogel composition onto a substrate (e.g., by extrusion through a syringe); and then

(c) optionally (as the secondary constructs may be produced by any suitable means) for general compositions and their tissue constructs, cross-linking the prepolymer with a second crosslinking group by an amount sufficient to increase the stiffness of said hydrogel and form said three-dimensional organ construct (e.g., by heating the hydrogel, irradiating the hydrogel composition with light (e.g., ambient light, UV light), altering the pH of the hydrogel; etc.); and

(d) for cardiac construct compositions, contacting the hydrogel with thrombin to cross-link the fibrinogen and form a fibrin hydrogel, as noted above.

The depositing step may be carried out with any suitable apparatus, including but not limited to 3d bioprinting techniques (including extrusion 3d bioprinting) such as that described in H.-W. Kang, S. J. Lee, A. Atala and J. J. Yoo, US Patent Application Pub. No. US 2012/0089238 (Apr. 12, 2012). In some embodiments, the depositing step is a patterned depositing step: That is, deposition is carried out so that the deposited composition is deposited in the form of a regular or irregular pattern, such as a regular or irregular lattice, grid, spiral, etc.

In some embodiments, the hydrogel composition containing cells is applied to the central region of a preformed 3D organoid substrate without the cells, resulting in distinct cell-containing zones (e.g., tumor cell-containing zones) inside of outer organoid zones.

In some embodiments, cell-free gelatin-only channels may be formed in the organoid substrate, forming channels in the construct that may aid in diffusion.

In some embodiments of general constructs, the cross-linking step increases the stiffness of said hydrogel by from 1 or 5 to 10, 20 or 50 kiloPascals, or more, at room temperature and atmospheric pressure. In some such embodiments, the hydrogel has a stiffness after said cross-linking step (c) of from 1 or 5 to 10, 20 or 50 kiloPascals at room temperature and atmospheric pressure.

In some embodiments, the method further comprises the step of depositing a supporting polymer (e.g., poly-L-lactic acid, poly(glycolic acid), polycaprolactone; polystyrene; polyethylene glycol, etc., including copolymers thereof such as poly(lactic-co-glycolic acid)) on said substrate in a position adjacent that of said hydrogel composition (e.g., concurrently with, after, or in alternating repetitions with, the step of depositing said hydrogel, and in some embodiments prior to the cross-linking step).

Any suitable substrate can be used for the deposition, including organic and inorganic substrates, and including substrates with or without features such as well, chambers, or channels formed thereon. For the particular products described herein, the substrate may comprise a microfluidic device having at least two chambers (the chambers optionally but preferably associated with an inlet channel and/or an outlet channel) connected by a primary fluid conduit through which the growth media may circulate, and the depositing is carried out separately in each chamber. In an alternative, the substrate may comprise a first and second planar member (e.g., a microscope cover slip), the depositing step may be carried out on that planar member, and the method may further comprise the step of inserting each planar member into a separate chamber of a microfluidic device. Post-processing steps, such as sealing of chambers, and maintaining the viability of cells, may be carried out in accordance with known techniques.

While the present invention is described primarily with reference to a single secondary chamber, it will be appreciated that multiple secondary chambers, with the same or different organoids, may be included on the substrate if desired. Thus the secondary chambers can be connected to one another, and the primary chamber, in any suitable configuration, including in series, in parallel, or in combinations thereof.

The substrate carrying the primary and secondary chambers, associated organoids, inlets, outlets, and conduits, may be provided in the form of an independent “cartridge” or subcombination that may be installed within a larger apparatus in combination with additional components for use. Thus, in some such larger apparatus embodiments, the apparatus further includes a pump operatively associated with the primary chamber for circulating the growth media from the primary chamber to the secondary chamber.

In some embodiments, the apparatus further includes (c) a cardiac monitor or beat monitor (e.g., a camera, electrode or electrode array, etc.) operatively associated with the cardiac construct (e.g., for monitoring the beat rate or frequency of the cardiac construct) and optionally operatively associated with the window.

In some embodiments, the apparatus further includes a growth media reservoir and/or bubble trap operatively associated with the primary chamber.

In some embodiments, the apparatus further includes a return conduit operatively associated with the primary and secondary chambers (and the pump, and reservoir and/or bubble trap when present) for returning growth media circulated through the secondary chambers to the primary chamber.

### D. Packaging, Storage and Shipping

Once produced, subcombination or “cartridge” devices as described above may be used immediately, or prepared for storage and/or transport.

To store and transport the product, a transient protective support media that is a flowable liquid at room temperature (e.g., 25° C.), but gels or solidifies at refrigerated temperatures (e.g., 4° C.), such as a gelatin mixed with water, may be added into the device to substantially or completely fill the chambers, and preferably also the associated conduits. Any inlet and outlet ports may be capped with a suitable capping element (e.g., a plug) or capping material (e.g., wax). The device may then be packaged together with a cooling element (e.g., ice, dry ice, a thermoelectric chiller, etc.) and all placed in a (preferably insulated) package.

Alternatively, to store and transport the product, a transient protective support media that is a flowable liquid at cooled temperature (e.g., 4° C.), but gels or solidifies at warmed temperatures such as room temperature (e.g., 20° C.) or body temperature (e.g., 37° C.), such as poly(N-isopropylacrylamide and poly(ethylene glycol) block copolymers, may be used.

Upon receipt, the end user may simply remove the device from the associated package and cooling element, allow the temperature to rise or fall (depending on the choice of transient protective support media), uncap any ports, and remove the transient protective support media with a syringe (e.g., by flushing with growth media).

### E. Methods of Use

An apparatus as described above may be used for screening at least one test compound for physiological activity, by:

(a) providing an apparatus as described above;

(b) optionally circulating a growth medium from the first chamber to the second chamber;

(c) administering at least one test compound to the constructs (e.g., by adding the test compound to the growth medium); and

(d) determining a change in beat frequency of the cardiac construct (e.g., with the cardiac monitor), typically as compared to that observed when the test compound is not administered.

In some embodiments, the at least one test compound comprise at least two distinct test compounds that are administered concurrently with one another, for example, to test for drug interactions therebetween.

In some embodiments, the determining step is carried out a plurality of times sequentially spaced from one another (e.g., at least two occasions spaced at least a day apart).

The methods and apparatus may be used, among other things, for the assessment of cellular metabolism, including metabolism of a particular test compound, or cellular toxicity induced by said by a particular test compound, or an interaction thereof.

Aspects of the present invention are explained further in the following non-limiting experimental examples.

### EXPERIMENTAL

In this study, we describe the development and testing of a liver and cardiac dual organoid-on-a-chip system for assessing physiological responses to drug and toxicology testing. To accomplish this, we have developed two types of high-functioning tissue organoids, which are integrated into a fluidic device system through hydrogel “bio-ink” and 3D bioprinting technology. Onboard this multi-organoid body-on-a-chip system, organoids are capable of responding to a variety of external stimuli independently or in a concerted manner,²⁰ similar to organ dynamics found in the human body, during which integrated biosensor systems can be employed for environmental and biological monitoring.

### Results Organoid Formation and Structural Characterization.

Liver organoids produced using the hanging drop culture method consistently formed uniform spheroidal aggregates of ˜250 μm in diameter and reliably remained+/−10 m throughout the 28 day culture period (data not shown). The initial seeding of 1,500 cells/organoid, with the specific mixture of cell types, reliably yields the desired diameter. Organoids were designed to maintain a size, dictated by cell number, that balances biological function with solute perfusion constraints that can cause hypoxia and the formation of a necrotic core.²¹

Histology of the liver organoids was used to examine the general organoid structure, organization of the different liver cell types, and formation of function-specific structures. Hematoxylin and eosin (H&E) staining (data not shown) shows compact organoid structure with thin, fibroblast-like cells lining the outside of the spheroid. Hepatocytes appear to be forming tight connections, as in native liver. Hepatocyte differentiation was analyzed by staining for albumin and cytochrome P450 reductase, showing widespread localization. Cytokeratin 18, a reliable marker for identification of human hepatocytes did not stain some cells along the outside of the structure, highlighting the fibroblast-like cells again (data not shown). GFAP, a marker for hepatic stellate cells, was only found in a few regions, congruent with desired proportions. Connexin 32 is a major gap junction protein expressed by hepatocytes, demonstrating that hepatocytes are forming structures important for long-term cell differentiation. E-cadherin staining reveals formation of cell-cell adhesion complexes between cells, suggesting hepatocyte polarity (data not shown).

Likewise, cardiac organoids were examined for several structural and functional markers via histological staining. Organoids positively expressed VEGF, which is expressed in 3D cardiomyocytes cultures, but not 2D cultures, suggesting improved capability to induce neovascularization,²² actinin, a microfilament protein required for attachment of actin to Z-lines of cardiac myofibrils, and cardiac troponin-T, a protein essential for cardiac muscle contraction (data not shown). Organoids expressed low levels of myosin regulatory light chain 7, which if expressed at higher levels would indicate regression of the cardiomyocytes to an immature state (data not shown). Interestingly, expression of MYL7 was only observed in node-like regions on the perimeter of the organoids. H&E staining showed a consistent distribution of cells throughout the interior of the organoids, as well as more diffuse aggregation compared to liver organoids (data not shown). Live/dead staining over various time points in culture demontrate high levels of viability (>95%) on day 1, day 28, and day 35 of culture (data not shown).

Liver Organoid Functional Characterization.

Liver organoid viability was monitored by measuring metabolism via a luminescent ATP assay at each time point, demonstrating that the organoids maintain viability for at least 28 days in culture (data not shown). The exact number of viable cells in culture cannot be accurately measured using this method because the cells included in the co-culture have different metabolic rates and their respective ratios are unlikely to remain consistent over time. However, this method does reliably allow for estimation of overall culture viability between time points. LIVE/DEAD staining provided similar evidence of viability (data not shown). This long-term maintenance of viability in three-dimensional spheroid cultured human liver co-cultures has been previously reported.²³

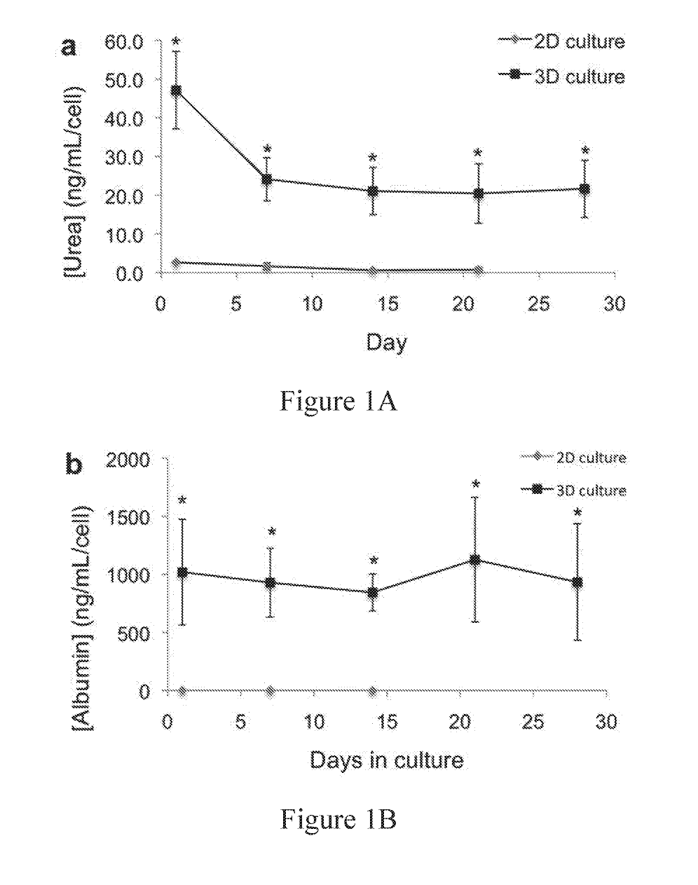

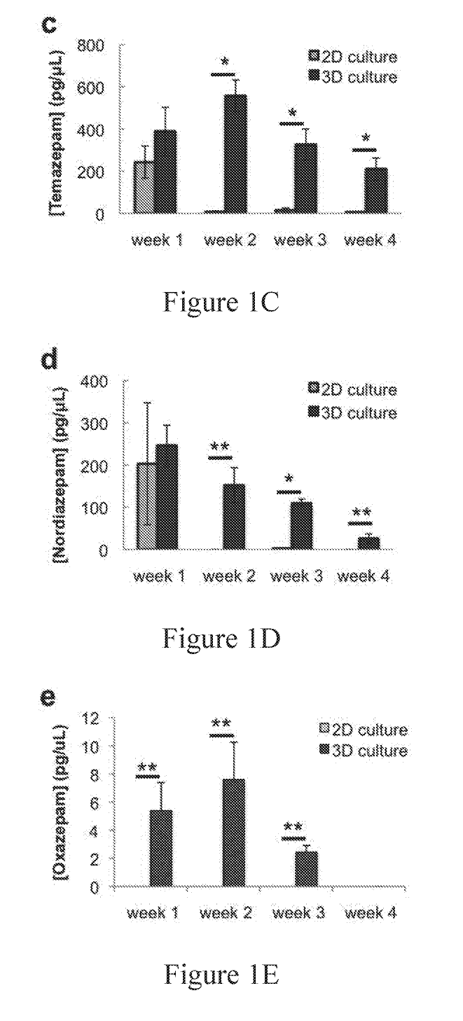

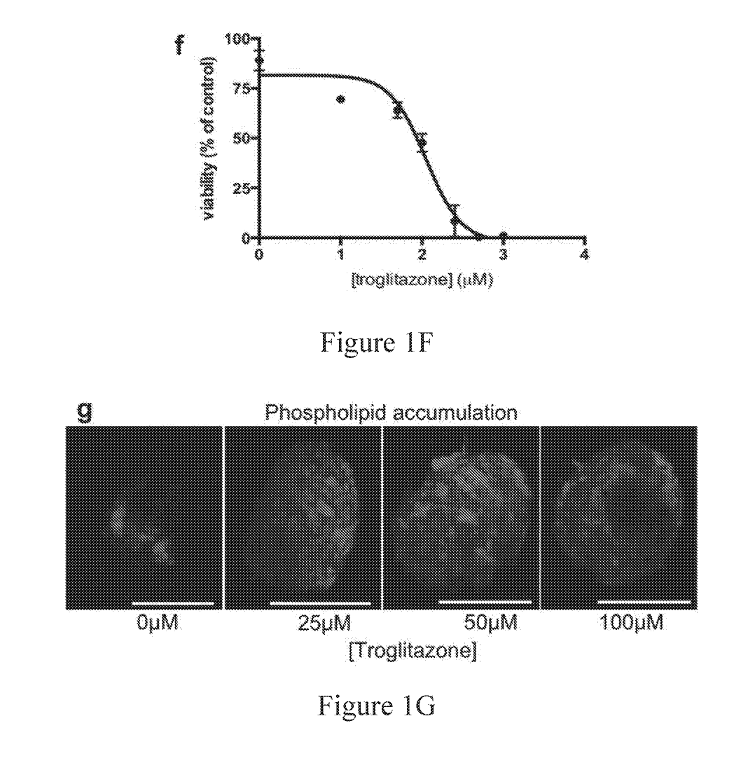

Liver organoid functionality initially was assessed by measuring urea and albumin production over time. Secretion of these compounds was maintained for at least 28 days in culture, suggesting long-term hepatocyte viability and functionality (FIG. 1a-b). Three-dimensional liver organoids produced significantly more urea and albumin than traditional monolayer cultures, despite containing fewer cells per culture (Liver organoid: ˜1,500 cells/sample. Monolayer cultures: ˜1,440,000 cells/sample). Monolayer cultures also failed to maintain measurable urea and albumin production after 21 and 14 days of culture, respectively. This long-term preservation of human hepatocyte viability and differentiation in spheroid form has been similarly reported by others.²³⁻²⁵

Liver-Specific Drug Metabolism and Drug Toxicity Response.

To evaluate drug metabolism capabilities, cytochrome P450 enzymes were induced using a series of compounds (rifampicin, 3-methylcholanthrene, and phenobarbital). Subsequently, the cells were exposed to diazepam, which is converted into primary metabolites temazepam and nordiazepam primarily by CYP3A4 and CYP2C19 (data not shown). A secondary metabolite, oxazepam can be further produced from the primary metabolites. The liver organoids were found to have measurable cytochrome P450 drug metabolism activity for at least 28 days in culture, in comparison to standard monolayer sandwich culture that lost CYP450 activity after 7 days (FIGS. 1c-e). This difference in performance between hepatocytes in spheroid culture versus hepatocytes in traditional monolayer has been previously reported.^{24,26} It is also important to note again the difference in total cell number between the 3D culture model (˜1,500 cells/sample) and the 2D culture model (˜1,440,000 cells/sample).

Trogligazone is a well-characterized hepatotoxic drug used to measure drug toxicity response in liver culture models. When liver organoids were treated with troglitazone for 48 hours, a dose-response curve shows a decrease in viability as concentration of drug is increased (FIG. 1f). Considerable phospholipid accumulation was found to occur in the organoids, even with lower concentrations of the drug (FIG. 1g).

Bioprinting Liver and Cardiac Organoids, Hydrogel Bioinks, Organoid Construct Design, and Integration into Fluidic System.

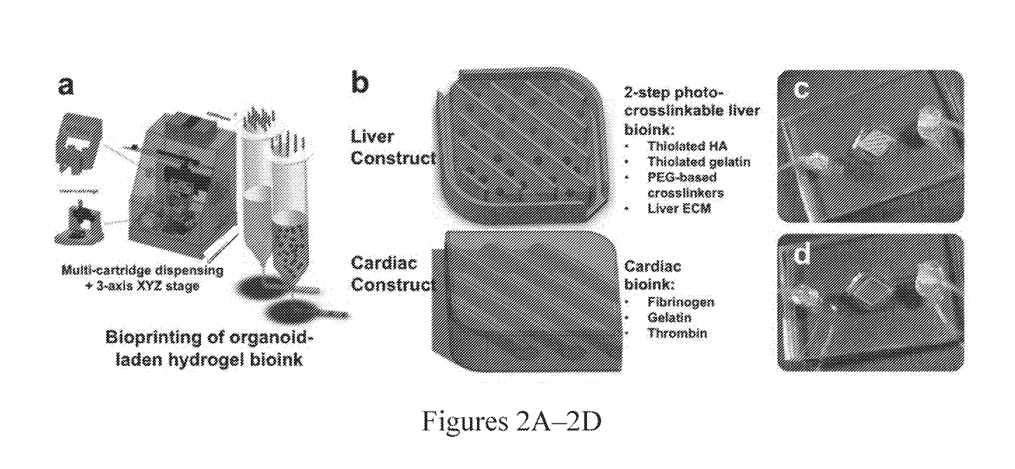

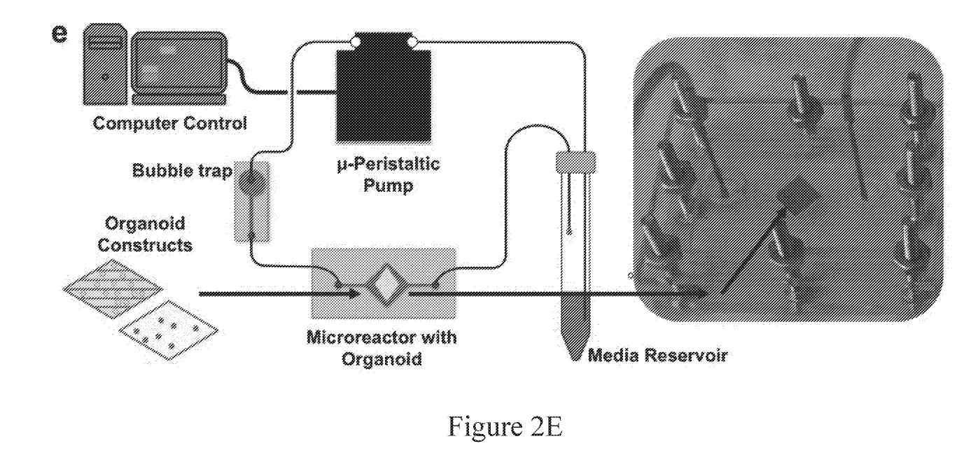

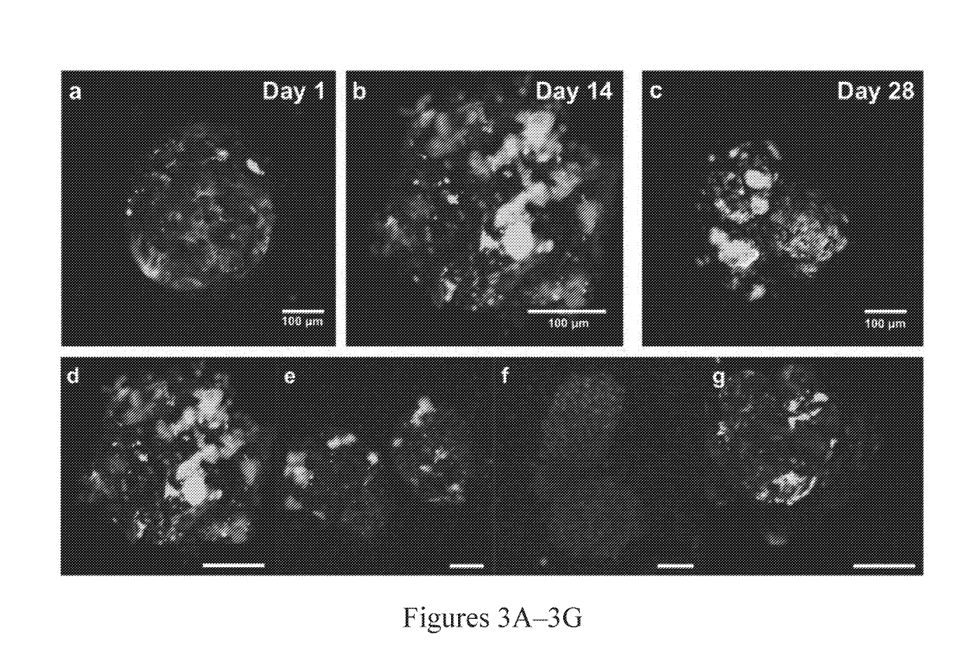

Bioprinting technology with X-Y-Z axis control and multiple print-heads, developed in house,²⁷ was employed (FIG. 2a) to create constructs comprised of 3D hydrogel microenvironments to house the organoids over longer-term cultures in the body-on-a-chip system (FIG. 2b-d) To accomplish this, hydrogel bioinks were developed that i) facilitated extrusion and ii) supported cellular viability and function. For liver constructs, the bioink was comprised of thiolated hyaluronic acid (HA), thiolated gelatin, liver extracellular matrix components,¹⁵ and a set of polyethylene glycol crosslinkers with acrylate or alkyne functional groups to facilitate a 2-step extrusion bioprinting protocol.²⁸ Unmodified HA and gelatin were supplemented to the bioink in order to ease the extrusion process. Organoids were suspended within the bioink, printed into the desired constructs, and UV light was employed to further crosslink the printed material to approximately the elastic modulus of native liver. The intensity of UV light employed here, and in other applications, has been previously demonstrated to be non-cytotoxic.^{29,30} Melt-cure extruded polycaprolactone filaments were printed alongside the bioink to act as a stabilizing support.

For cardiac constructs, the bioink was comprised of 2 parts: i) fibrinogen and gelatin, and ii thrombin. Organoids suspended in the fibrinogen-gelatin mixture were printed onto a cool stage (20° C.) to maintain the gelled state of the gelatin, after which thrombin was printed over the construct to induce the formation of fibrin. Cell-free gelatin-only channels were incorporated into the 3D space of the cardiac constructs to aid with diffusion. These constructs were bioprinted onto coverslips for integration into microfluidic devices. In general, bioprinted liver constructs contained 45-50 liver organoids, while cardiac constructs contained 9-11 organoids, a ratio reflecting mass of liver and heart in humans.

Microfluidic devices (also called microreactors) consisted of individual units with chambers for organoids, each accessible via a fluidic channel with individually addressable inlets and outlets connected to a micro-peristaltic pump for driving flow through parallel circuits (FIG. 2e). These devices are fabricated using conventional soft lithography and replica molding.³¹ Integration of organoids with the microreactor devices supporting microfluidic fluid flow primarily relied on the ability to immobilize the organoids inside the microreactor organoid chamber. If they were not held in place, individual spherical organoids could be pulled into circulation and become obstructions in the microfluidic channels and tubing, thereby impeding media flow through the entire system. Fortunately, in addition to facilitating bioprinting and supporting organoid function, the hydrogel bioinks served as effective organoid immobilizing agents. Organoid constructs on the 7 mm by 5 mm diamond-shaped coverslips were plugged into the microreactor organoid chambers. The close fit ensured that the constructs stayed in the bottom of the chambers. The spherical organoids remained encapsulated within the hydrogels, and problems due to clogging by organoids were avoided. FIG. 2e depicts the integration of a bioprinted liver organoid structure with the microreactor device.

Liver Constructs Maintain Viability, Phenotype, and Function, and Respond to Toxins in a Physiological Manner Onboard Fluidic System Culture.

Liver organoids in hydrogel constructs in the microfluidic system were assessed independently from cardiac organoids for initial system characterization. After 8-day microreactor cultures, organoids were fixed and stained using immunofluorescence to assess a panel of structural and functional markers (data not shown). The organoids stained positive for CYP3A7, an enzyme in the cytochrome p450 family involved in drug metabolism, and albumin, which together demonstrate maintenance of liver function (data not shown). Additionally, some cells expressed OST-α, a basolateral transporter, and dipeptidyl peptidase IV (DPP-4), an apical membrane protein, suggesting polarity within the hepatocytes (data not shown). Furthermore, the liver cells express membrane-bound ZO-1, a tight junction marker, as well as E-cadherin and ß-catenin, demonstrating appropriate epithelial-like cell-cell organization (data not shown). Together, these images indicate that the liver organoids are capable of expressing a number of important proteins critical to functional liver tissue, and importantly, these proteins continue to be expressed after the organoids are removed from traditional culture settings, and integrated into a microfluidic platform a described above.

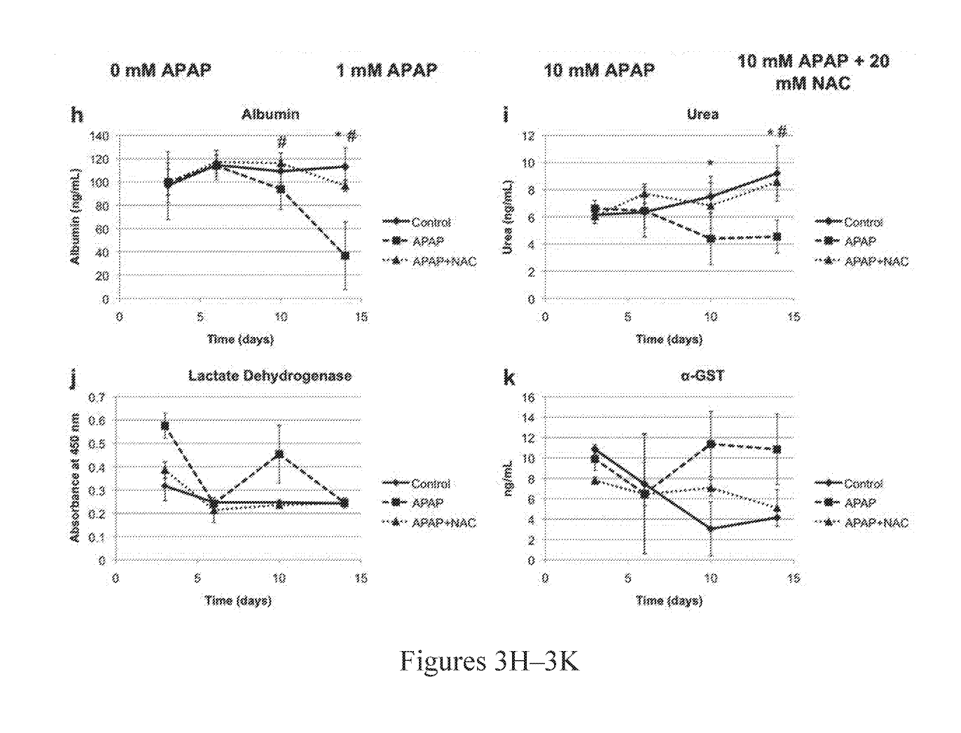

Bioprinted liver organoids were further cultured in microreactors for up to 28 days, during which time sets of organoids were removed from culture on day 1, day 14, and day 28 for assessment of viability. Viability was assessed qualitatively by LIVE/DEAD staining and whole-mount microscopy. FIG. 3a-c shows representative images of LIVE/DEAD-stained liver organoids removed from microreactor culture on day 1, day 14, and day 28. The images show a high percentage of viable cells stained green by calcein AM. At each time point there were observed to be dead cells present, stained in red by ethidium homodimer, but in general these are fewer in number.

To demonstrate clinical relevance, liver construct response to toxicity was assessed by treatments of acetaminophen (APAP) and by the clinically used drug N-acetyl-L-cysteine (NAC). Liver constructs in the fluidic system received no drug, 1 mM APAP, 10 mM APAP, or 10 mM APAP+20 mM NAC. Viability was assessed by LIVE/DEAD staining and whole-mount imaging. Based on the ratio of live (green) cells to dead (red) cells, it was evident that the 0 mM control group maintained a relatively high level of viability (70-90% at day 14) throughout the 14 day experiment (FIG. 3d). In comparison, the 1 mM APAP group had decreased viability (30-50% at day 14, FIG. 3e), while the 10 mM APAP group appeared to have few viable cells at day 14 (FIG. 3f). Treatment with NAC reduced the level of morbidity associated with the high concentration of APAP, and instead, organoids appeared more like those that received the lesser 1 mM APAP treated organoids (FIG. 3g). Albumin analysis revealed constant albumin production by liver organoids through day 6, remaining on average near 120 ng/mL (FIG. 3h). Albumin levels at the first two time points were not statistically significant in comparison to one another, as would be expected as no drugs had been administered at this point. Following APAP administration after day 6, albumin levels were significantly decreased in both the 1 mM and 10 mM groups compared to the 0 mM control (p<0.05). Additionally, the 10 mM group albumin levels were significantly decreased compared to the 1 mM group (p<0.05). At day 14 the albumin levels in the 10 mM group were nearly immeasurable. Albumin levels in the APAP+NAC organoid were significantly greater than those of the 10 mM APAP treated group. The general trend of the data was appropriate, suggesting that the liver organoids respond to APAP correctly, and can be rescued by NAC, as patients in the clinic might be. Urea analysis also showed results with similar trends (FIG. 3I). Urea levels were not significantly different between groups during the time points prior to APAP administration. After APAP administration, measured urea levels appeared to drop in a dose dependent manner with respect to APAP concentration. On the day 10 time point, the 0 mM control group albumin level was significantly higher than both the 1 mM and 10 mM group (p<0.05). On the day 14 time point, these three groups were significantly different from one another (p<0.05). The APAP+NAC organoid urea levels were not significantly different than the control organoids, but were significantly greater than the 10 mM APAP urea levels (p<0.05).

Media samples were then analyzed for lactate dehydrogenase (LDH) and α-glutathione-S-transferase (α-GST) (FIG. 3j-k), which when released from liver cells are indicators of cell death. There is initial variability in LDH levels on day 3. This could be attributed to stresses placed on the cells during the bioprinting and microfluidic initiation phases of the cultures. By day 6, all groups are indistinguishable from one another. On day 10, the first collection point after drug administration, the 10 mM APAP group shows a clear increase in LDH concentration in the media, while the APAP+NAC group is almost identical to the control group. The APAP group is not significantly different from the other groups on day 10, but the trend is evident. By day 14, the LDH levels drop down to baseline, suggesting the majority of LDH release occurred between day 6 and day 10, resulting in the spike in quantified LDH on day 10 in the APAP group. The organoids in each group had secreted similar levels of α-GST at the day 3 and day 6 time points. Detectable levels of α-GST (between 7 and 11 ng/mL) were present on day 3 in all groups, which then decreased over time in the control group. Again, this suggests that the bioprinting process and initiation of microfluidic culture may have placed some stress on the cells in the organoids, resulting in some cell death at the outset of the microreactor cultures. After administration of 10 mM APAP, α-GST increases to over 11 ng/mL by day 10, and stays near that level until the end of culture. In comparison, in the control organoid group α-GST decreased to less than 4 ng/mL, indicating that APAP does indeed invoke cell death resulting in release of α-GST into the media. Administration of NAC with APAP clearly attenuated the effects of APAP. On day 10 and day 14, α-GST was detected at about 6 and 5 ng/mL, respectively, in APAP+NAC cultures.

Cardiac Constructs Support Baseline Function and Response to Beat Rate-Altering Drugs.

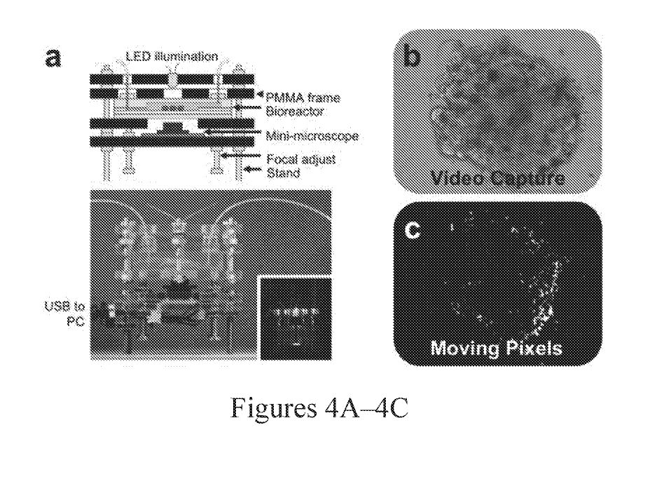

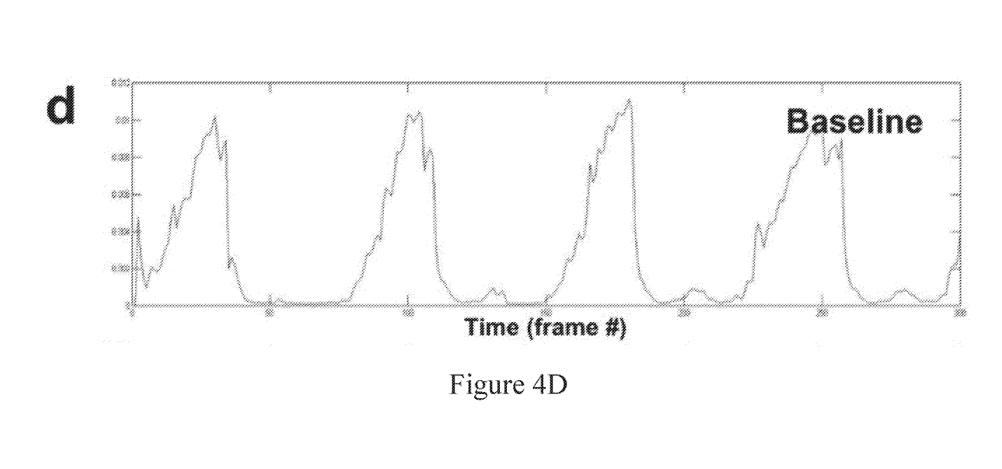

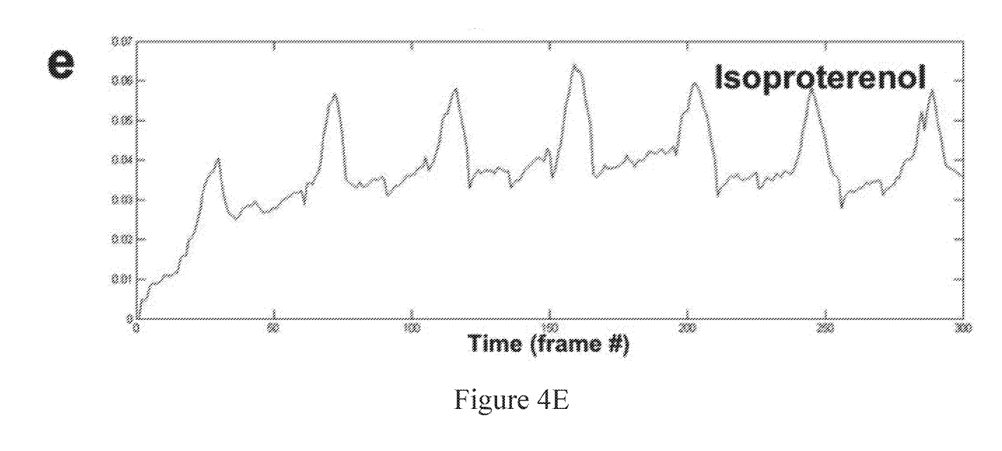

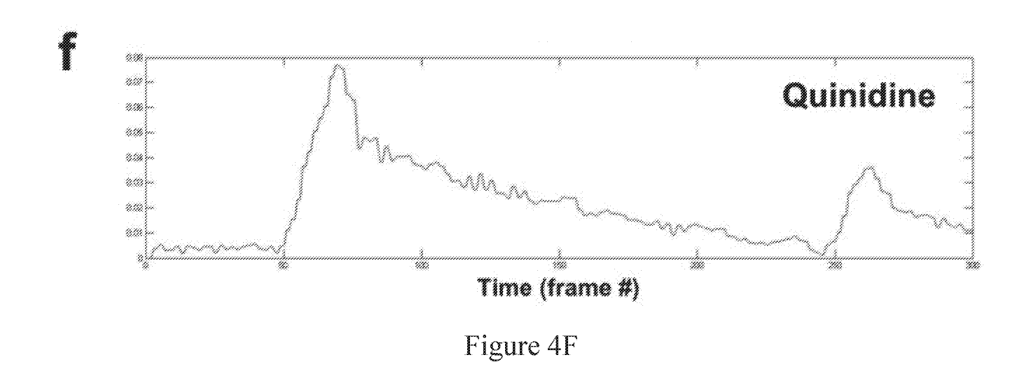

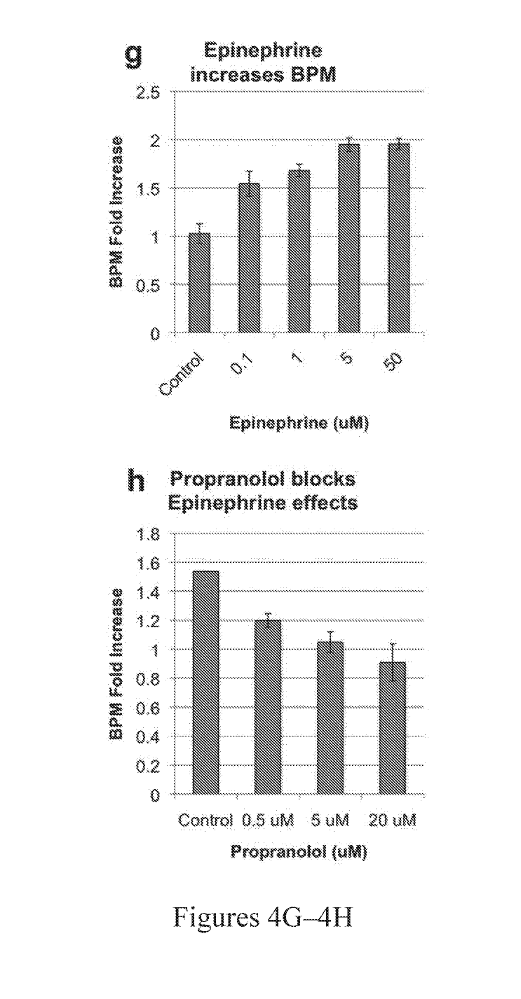

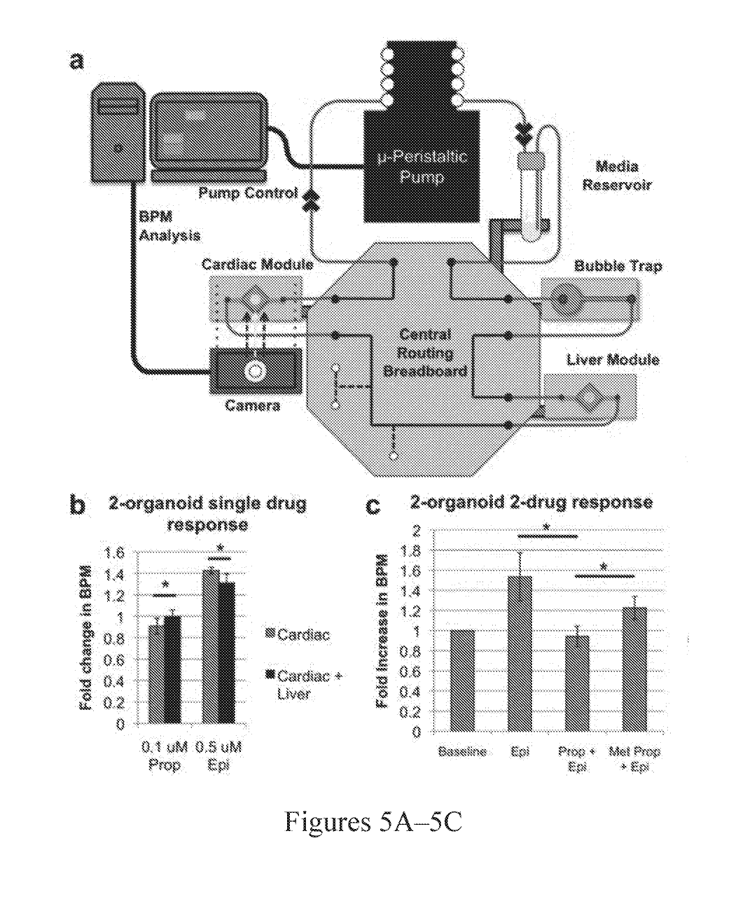

Since one of the primary output metrics for cardiac constructs is quantification of beating, real-time visual monitoring of cardiac organoids was achieved using an onboard LED and camera system that was customized to integrate with the cardiac construct microreactor housing (FIG. 4a). This system allowed video capture capability at will, which provided video files of cardiac organoids beating in real time (FIG. 4b). Using custom written MatLab code with a series of MatLab functions, moving pixels in each frame were determined over time, generating a binarized representation of beat propagation (FIG. 4c) and a plot visualizing beating rates. An example of a beat plot under baseline conditions is shown in FIG. 4d.

A necessary feature of engineered cardiac constructs is the ability to respond in a physiologically accurate manner to drugs and other external stimuli. A variety of heart beat-modulating drugs were administered to the cardiac constructs during which the change in beating behavior was captured as described. Isoproterenol (0.1 mM), a beta-adrenergic agonist often used to treat patients with bradycardia, increased organoid beating rate (FIG. 4e). Conversely, quinidine (1 μM), an ion channel blocker that slows depolarization and repolarization and is used as an anti-arrhythmic drug, slowed organoid beating rate as expected (FIG. 4f).

Additionally, physiologically relevant concentrations of epinephrine and propranolol were assessed for their efficiency at inducing and preventing cardiac organoid beating rate increases. First, five epinephrine concentrations (0, 0.1, 0.5, 5, and 50 uM) were tested on cardiac organoids to determine the lowest concentration that initiates a clearly discernable faster beating rate. Beating rates of organoids were measured before and after epinephrine administration. Organoid beating increased in a dose-dependent manner, until plateauing after 5 uM, likely due to saturation of beta adrenergic receptors (FIG. 4g). Next, four propranolol concentrations (0, 0.5, 5, and 20 uM) were administered to cardiac organoids. Organoids were incubated under these conditions for 20 minutes, after which epinephrine was then added at 5 uM. In general, increasing concentrations of propranolol incubation more effectively prevented epinephrine-induced increases in beating rates (FIG. 4h), demonstrating an appropriate beta blocking response in the presence of epinephrine.

Liver Metabolism in a Dual Organoid Liver and Cardiac Platform Influences the System Response to Drugs.

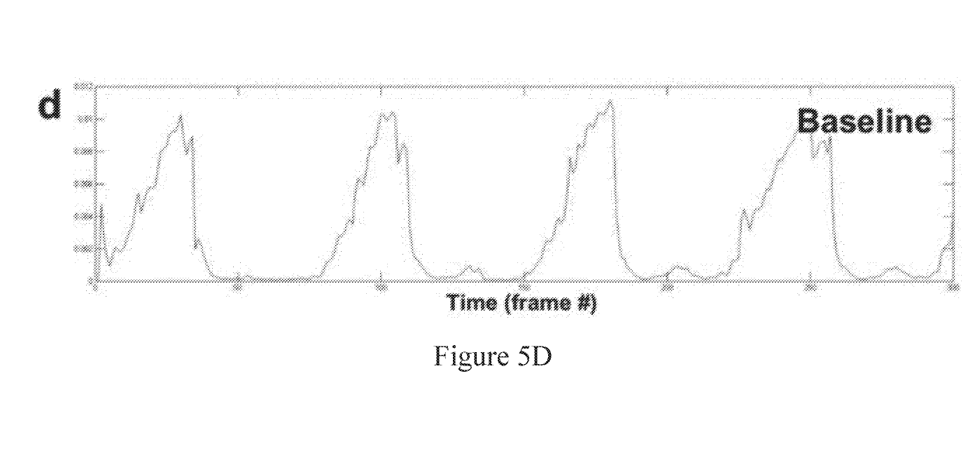

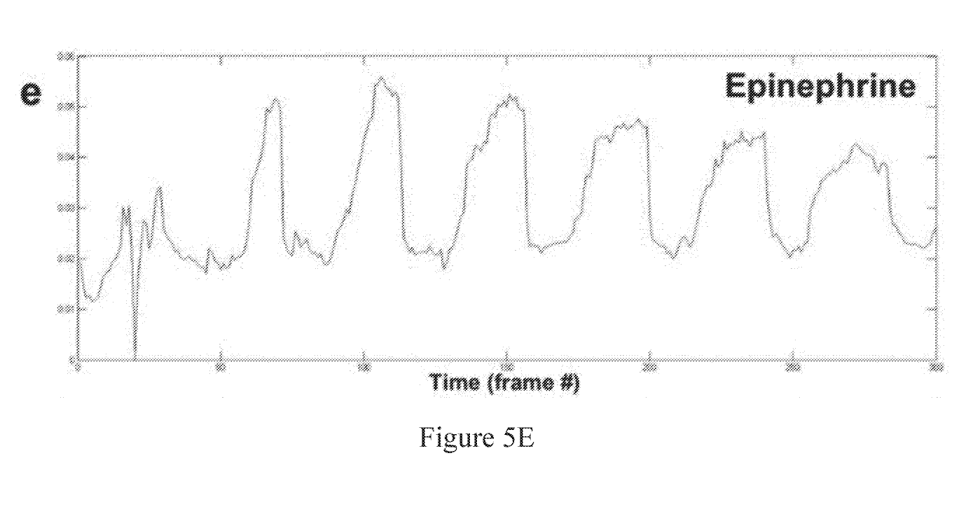

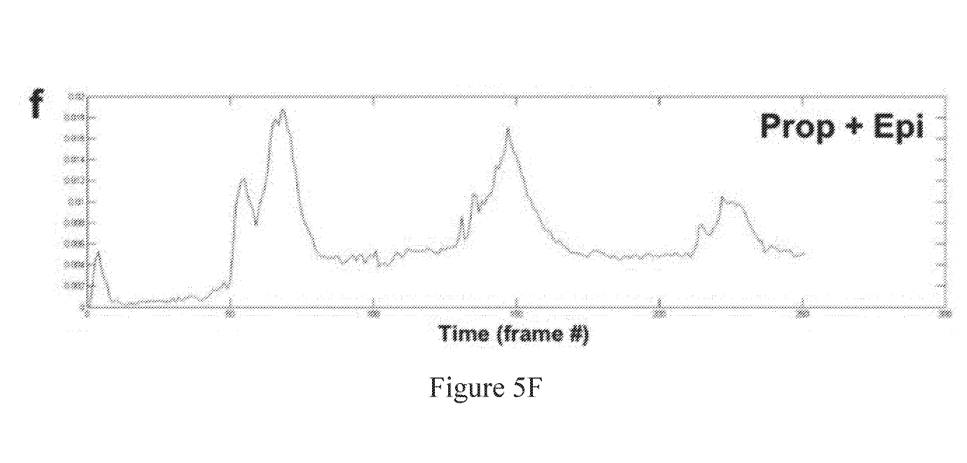

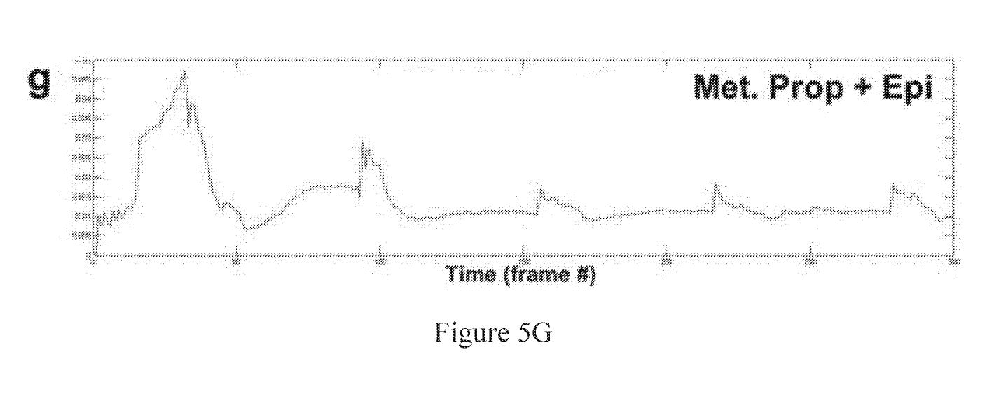

In the human body, organs interact with one another in complex ways. To demonstrate that the organoid platform can also support multi-organoid interactions, experiments were performed in which the functionality of the downstream cardiac construct was dependent on the upstream liver construct metabolism. The modular nature of the fluidic system was employed to realize such a platform. A central fluid-routing breadboard comprised of PDMS was used to direct flow of a common media from the μ-peristaltic pump and media reservoir through a bubble trap, the microreactor containing a liver construct, the microreactor containing the cardiac construct with the integrated onboard camera system, and back to the pump (FIG. 5a). Additional optional ports are depicted in FIG. 5a that were not employed in these experiments, but allow for further customization of the system.

To assess the impact of combining the two tissue construct types in one system, effects of epinephrine and propranolol were first tested independently with a cardiac-only system or the tandem system, before being tested jointly in both systems. Treatment with propranolol only (0.1 μM) resulted in a small (˜10%), but significant (p<0.05) fold decrease in beating rate in the cardiac-only system. However, in the presence of the liver construct, there was no decrease in beating rate, indicating some metabolism of the drug (FIG. 5b). Similarly, treatment with epinephrine only (0.5 μM) resulted in a significant (˜40%) fold increase in beating rate in the cardiac-only system. Addition of the liver component did not negate the epinephrine induced beating rate increase, but reduced the increase from approximately 40% to 30% (p<0.05, FIG. 5b), further demonstrating the integrated organoid system response.

Next, the interplay between both drugs in the cardiac-only and tandem systems was assessed. Drug concentrations of 0.1 μM propranolol and 0.5 μM epinephrine were chosen based on the results described above (FIG. 4g-h). Propranolol was administered first after which epinephrine was subsequently added, and depending on which organoids were present and functioning, the effect of epinephrine would vary (FIG. 5c). Beating plots for each condition are depicted in FIGS. 5d-g. In Group 1, which did not have liver organoids, 0.1 μM propranolol remained active, and successfully blocked the beta-adrenergic the effects of 0.5 μM epinephrine. This was expected as there was no liver component to metabolize the blocking agent. In Group 2, in which the liver component was added, after the epinephrine was administered, a 1.25 fold increase in BPM was observed. This was compared to a 1.5 fold increase in cardiac BPM in experimental controls where no propranolol was administered prior to epinephrine treatment. This suggests that the 3D liver organoids metabolized enough of the propranolol so that epinephrine could activate a significant percentage of the beta adrenergic receptors of the cardiac organoids, inducing the equivalent of approximately 50% of the control epinephrine-only response, highlighting the effect that multiple organoid systems have compared to single organoid systems. Interestingly, conditions in Group 2 were repeated using a 2D hepatocyte culture comprised of 1-2 million cells on tissue culture plastic versus the 50,000 cells making up the 3D organoids within the microreactor. The 2D cultures failed achieve any restoration of the epinephrine-induced increase in beat rate, further suggesting the lack of sufficient metabolic activity in 2D cultures compared to 3D systems.

Integrated Biosensing System.

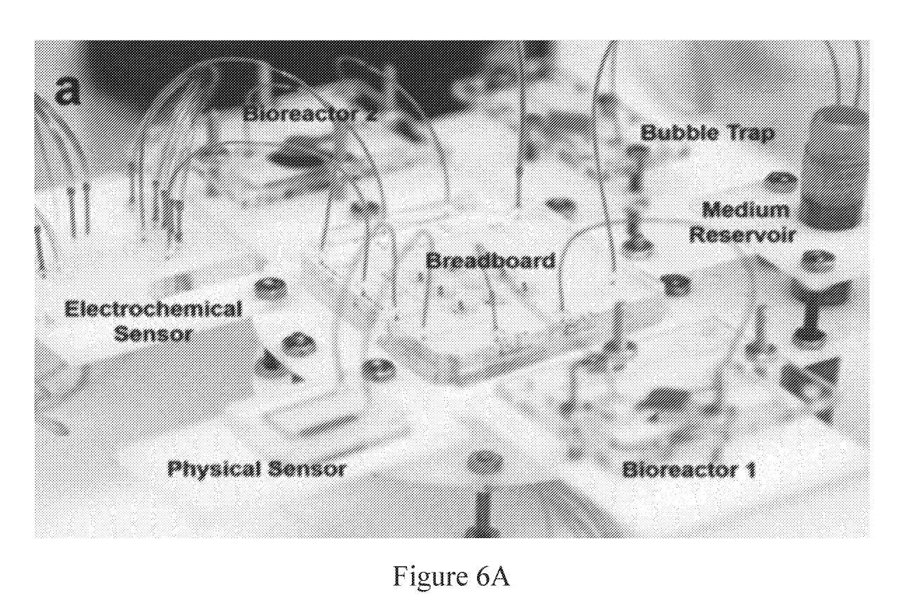

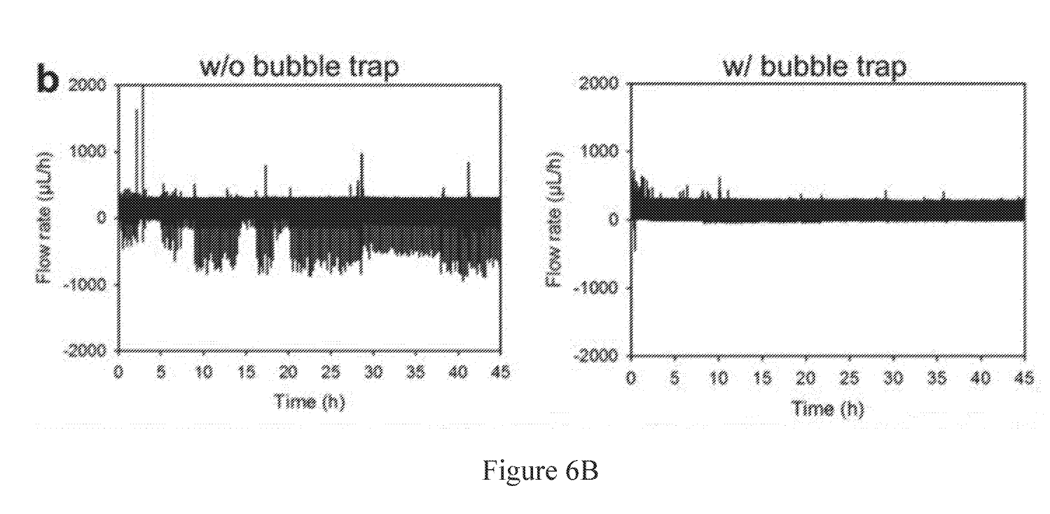

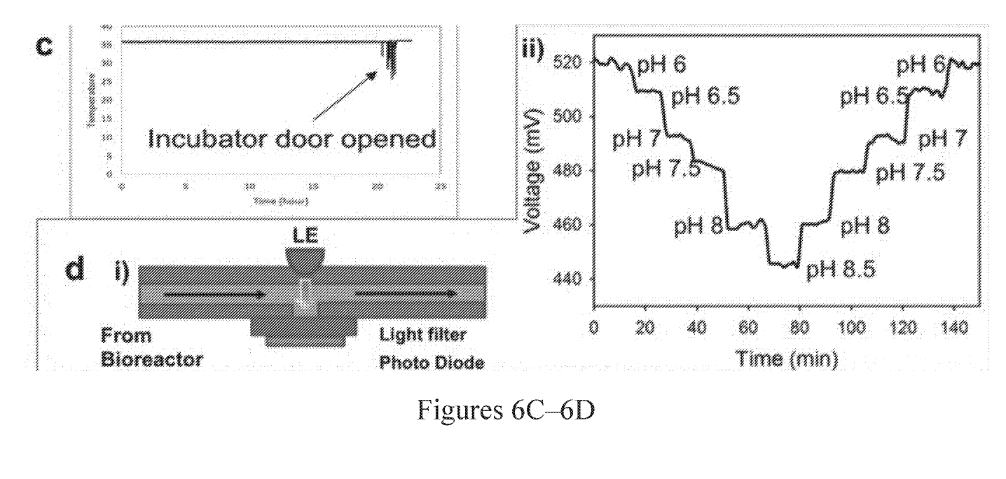

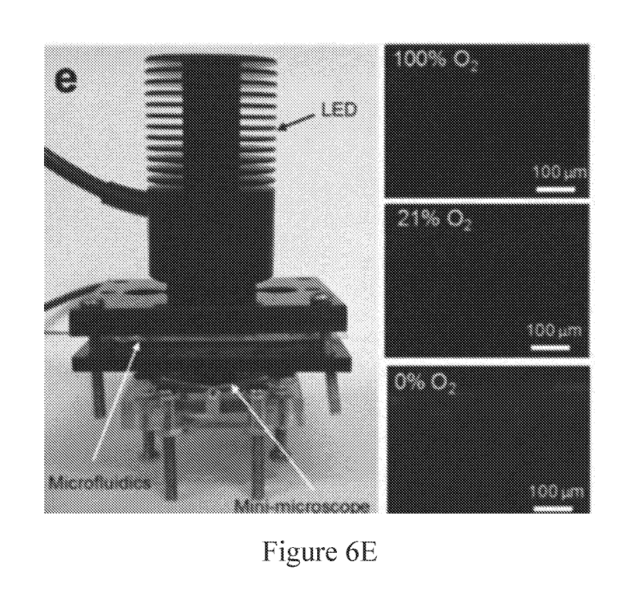

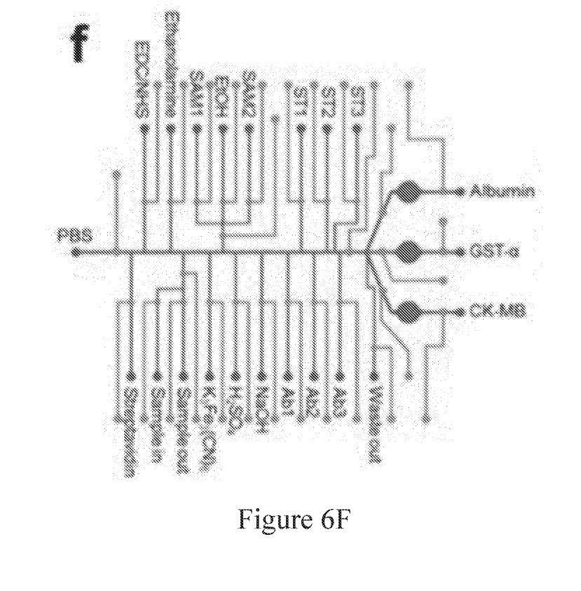

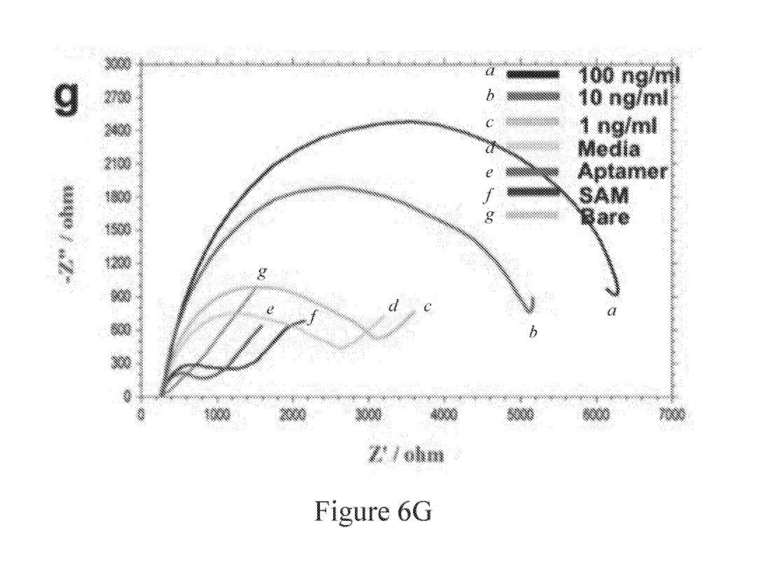

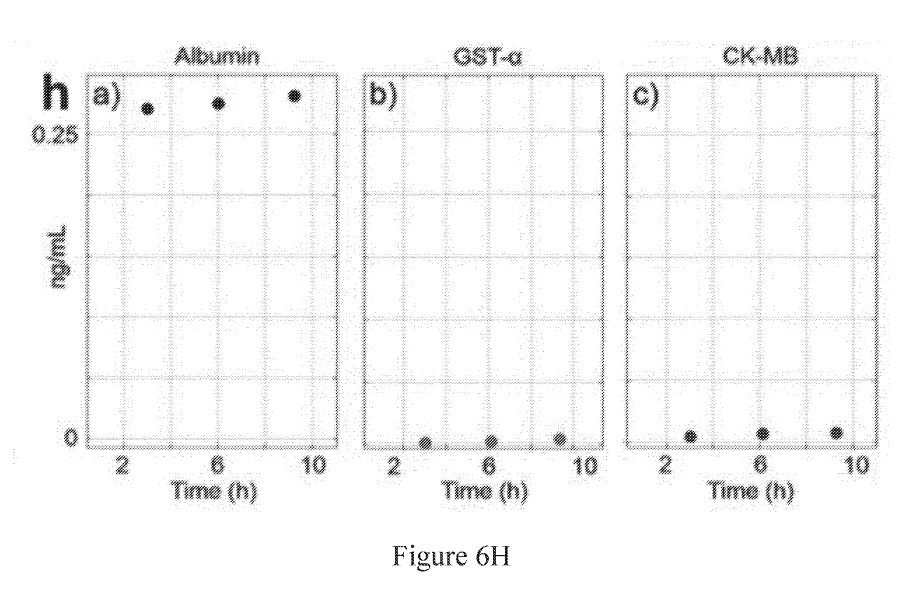

The preceding data demonstrate the potential that a systems biology approach to an in vitro organoid platform can have. However, from an analytical point of view, with the exception of the cardiac beating activity monitoring, the data output is still in the form of snapshots at a relatively small number of time-points achieved by established, but often tedious, traditional techniques such as ELISAs and immunostaining. To improve on these standard measurement techniques, sensors were combined with the microfluidic components to create a system comprised of the central breadboard for routing fluid flow to outside components, a media reservoir, a bubble trap, multiple organoid microreactors, a physical sensor chip, and an electrochemical sensor chip (FIG. 6a). The integrated bubble trap is comprised of a module through which media flow encounters a grid of posts, which serve to capture and consolidate bubbles, at which point they can be removed from the system as desired.³² Testing with an inline Mitos flow sensor shows fluctuations in flow rate without the bubble trap compared to more uniform and consistent flow with the bubble trap (FIG. 6b). A physical sensor module houses 3 sensors: a temperature probe, a pH sensor, and an oxygen sensor. The thermocouple temperature probe records the temperature of the passing media flow, and responds to perturbations in the environmental temperature, as demonstrated by opening the incubator door and allowing ambient room temperature air in (FIG. 6c). Media pH and oxygen sensors are based on inline LED and photodiode systems, and are particularly sensitive to physiological value ranges, such as pH 6.0 to 8.5 (FIG. 6d) and 0% to 21% O₂ (FIG. 6e).³³ Finally an electrochemical sensor module based on antibody or aptamer binding and changes in electrode impedance provides intermittent measurements of up to three soluble biomarkers at a time over the course of system operation (FIG. 6f-g). An operational integrated system was constructed which recorded electrochemical biomarker data over the course of a 12-hour cycle for tissue construct-secreted albumin, α-GST, and creatine kinase. Albumin levels are measurable and consistent, while α-GST and creatine kinase remain low, as under these baseline conditions no toxicity was expected (FIG. 6h).

### Discussion

Development of effective new drug candidates has been limited and made incredibly expensive due to the failure to accurately model human-based tissues in vitro. Animal models allow only limited manipulation and study of these mechanisms, and are not necessarily predictive of results in humans. Traditionally, in vitro drug and toxicology testing has been performed using cell lines in 2D cultures. Despite having yielded many discoveries in medicine, 2D cultures fail to accurately recapitulate the 3D microenvironment of in vivo tissues.⁵⁷′⁸ By transitioning to 3D tissue organoids, many of these shortcomings can be overcome. 3D organoids, while small in size, have diffusion characteristics more like those of in vivo tissues, as well as allowing many of the naturally occurring cell-cell and cell-matrix interactions to form. Such organoids have dramatically improved tissue-specific functionality compared to their 2D counterparts, as we have shown in our organoid characterization data. More importantly, these organoids have the capability to respond to drugs and toxins in the same manner as actual human organs do, and as such, they provide an improved platform for drug screening applications.

We further describe the integration of these liver organoids and cardiac organoids with bioprinting and microfluidic technology, ultimately resulting in a multi-organoid system that responds to a range of drugs. First, we assessed liver and cardiac organoids separately. Acetaminophen, a common liver toxin when taken in large doses, was shown to decrease both liver organoid-secreted albumin and urea in a dose dependent manner. Additionally, LIVE/DEAD viability assessment showed that increasing APAP doses caused a clear increase in cell death. These responses were expected, and suggested that these in vitro bioprinted organoids respond as they should to APAP. The next experiments focused on using N-acetyl-L-cysteine as a counteracting agent to mitigate the toxic effects of APAP. Administration of APAP with concurrent NAC treatment reduced the toxic effects, resulting in functional output that more closely resembled the no drug control groups. NAC mitigated the APAP-induced decrease in albumin and urea output, and also decreased the incidence of LDH and α-GST release from apoptotic cell death, thus demonstrating the responsiveness of the liver organoids not only to toxic drug doses, but to rescuing agents. Responsiveness of cardiac organoids was tested using epinephrine, a beta-adrenergic agonist, and propranolol, a beta-blocker. Activation of beta-adrenergic receptors by epinephrine normally results in increased beating rates, while propranolol blocks this effect. A range of epinephrine concentrations were tested, resulting in organoid beating that increased in a dose-dependent manner, until eventually plateauing, likely due to saturation of beta adrenergic receptors. When propranolol was administered prior to a high concentration dose of epinephrine, cardiac beating rate increases could be decreased, or blocked, in a dose-dependent manner. Importantly, responses to epinephrine were rapid, despite the low fluid flow rates in the system, suggesting that it may be possible to achieve near physiological response rates to various drugs.

More important than individual organoid responses to drugs is a multi-organoid system response, in which the responses of one organoid have implications on the responses of other organoids. To explore this concept, liver and cardiac organoids were combined within single circulating fluid systems. Since native, healthy liver can efficiently metabolize propranolol, rendering it ineffective at blocking beta-receptors, the effects of propranolol blocking and epinephrine-based beta receptor activation was evaluated with and without liver organoids. In systems with no liver organoids, propranolol remained in its active form within the system and successfully blocked epinephrine from inducing beating rate increases. However, when 3D liver organoids were introduced, they metabolized some of the propranolol, and upon administration of epinephrine, beating rates increased, indicating significant liver metabolism. Notably, if hepatocyte cultures in 2D were substituted for the 3D liver organoids, propranolol blocked epinephrine's effects as if no liver cells were present at all. This further validated the liver organoid platform, demonstrating the importance of 3D tissue organization.

In addition to the necessity to maintain high levels of cell viability and function in 3D in vitro screening platforms, there is also a need for improved data acquisition systems. Even the most advanced biological platforms will not gain widespread use if the acquisition and monitoring technologies are not simple to operate or comprehensive. The meet these requirements, our team developed a portfolio of sensing systems, that like the other components of the platform are modular in nature, allowing rapid implementation in a plug-and-play manner. Demonstrated are a set of physical environment sensors, including temperature, flow rate, oxygen, and pH, and cell-based sensors, including onboard cameras for capturing cardiac organoid beating and advanced antibody or aptamer-based electrochemical sensors for monitoring soluble biomarker concentrations. Further integration and streamlining of these sensors with the tissue construct units and fluidic components will continue to advance the utility of our platform, and support low reagent and sample consumption, short assay times, and low operating cost.³⁴

### Methods

Organoid Production and Maintenance.

Organoids were aggregated using GravityPlus hanging drop culture plates (inSphero AG). The cells were combined in a cell seeding mixture comprised of 90% HCM medium (Lonza), 10% heat-inactivated fetal bovine serum (Gibco), and rat tail collagen I (10 ng/μl, Corning). Liver organoids were produced with a mixture of 80% hepatocytes (Triangle Research Labs), 10% hepatic stellate cells (ScienCell), and 10% Kupffer cells (Gibco). Approximately 1500 cells per 40 μL media were used to form aggregates in hanging drop culture. Cardiac organoids were produced similarly in cardiomyocyte maintenance medium (Stem Cell Theranostics) with 100% cardiomyocytes (Stem Cell Theranostics) to maintain culture purity and differentiation. After 4 days of culture at 37° C. with 5% CO₂, the organoids were transferred for downstream applications and cultured in their respective culture media at 80 l/well.

Liver- and Cardiac-Specific Hydrogel Bioink Preparation.

Liver-specific hydrogel bioinks were formulated using a hyaluronic acid and gelatin hydrogel system infused with a liver ECM solution, containing growth factors, collagens, glycosaminoglycans, and elastin, which was prepared from decellularized porcine livers as described previously.¹⁵ For bioink preparation, the thiolated hyaluronic acid and gelatin base material components from HyStem-HP hydrogel kits (Heprasil and Gelin-S, respectively, ESI-BIO, Alameda, Calif.) were dissolved in a 0.1% w/v solution of photoinitiator (4-(2-hydroxyethoxy)phenyl-(2-propyl)ketone, Sigma) to make 2% w/v solutions. A PEGDA crosslinker (MW 3.4 kDa, ESI-BIO) was dissolved in the phoinitiator solution to make a 4% w/v solution. Additionally, an 8-arm PEG Alkyne crosslinker was dissolved to make an 8% w/v solution. To prepare the hydrogel bioink solution, 4 parts 2% Heprasil, 4 parts 2% Gelin-S, 1 part crosslinker 1, 1 part crosslinker 2 is combined with 8 parts liver ECM solution and 2 parts Hepatocyte Culture Medium (HCM, Lonza). Unmodified HA and gelatin was then supplemented to the bioinks (1.5 mg/mL and 30 mg/mL, respectively). The resulting mixture was vortexed to mix, transferred into a syringe or printer cartridge, and allowed to crosslink spontaneously for 30 minutes (stage 1 crosslinking). When secondary crosslinking (stage 2) was desired, for example, after bioprinting, the extruded stage 1-crosslinked gels were irradiated with ultraviolet light (365 nm, 18 w/cm²) to initiate a thiol-alkyne polymerization reaction.

Cardiac hydrogel bioinks were formulated using a simple fibrin-gelatin 2-part system. The first part was prepared by dissolving 30 mg/mL fibrinogen and 35 mg/mL gelatin in PBS, while the second part was prepared by 20 U/mL thrombin in PBS. Crosslinking of the bioink components into a hydrogel was achieved by covering the desired volume of the fibrinogen-gelatin solution with the thrombin solution, thereby initiating enzymatic fibrinogen cleavage and subsequent crosslinking.

Liver Construct and Cardiac Construct Bioprinting.

To fabricate liver constructs, primary liver spheroids were suspended within the hydrogel bioink solution, transferred to a bioprinter cartridge, after which the solution was allowed to undergo the first crosslinking stage (thiol-acrylate reaction) for 30 minutes. Following initial crosslinking, a 3D bioprinter developed in house²⁷, was employed to extrude the hydrogel bioink concurrently with polycaprolactone to form a set of hydrogel “channels” between supportive PCL structures on top of a 7 mm by 5 mm diamond-shaped plastic coverslip. This architecture is described in FIG. 2b-c. Printing was performed under 20 kPa pressure applied by the bioprinter while the printhead moved in the X-Y plane at a velocity of approximately 300 mm/min. After deposition, administration of UV light for 1-2 seconds was used to initiate the secondary crosslinking mechanism, stabilizing the constructs and increasing material stiffness. Constructs were placed in the bottom of 12-well plates, covered with 2 mL HCM, and plates were placed in an incubator at 37° C., 5% CO₂ until further use.

To fabricate cardiac constructs, cardiac organoids were suspended within the fibrinogen-gelatin solution, and transferred to a bioprinter cartridge. The gelatin component added sufficient viscosity to the bioink, holding the organoids in suspension and facilitating smooth deposition. The 3D bioprinter deposited the organoid-laden bioink within a supporting PCL frame located along the perimeter of the same 7 mm by 5 mm plastic coverslips described above. Printing was performed as described above, after which the secondary solution of thrombin was used to cover the bioprinted construct, initiating crosslinking of the fibrinogen component. Constructs were placed in the bottom of 12-well plates, covered with 2 mL CMM with 20 μg/mL aprotinin (Sigma) to prevent enzymatic breakdown of the fibrin gel, and well plates were placed in an incubator at 37° C., 5% CO₂ until further use.

For verification of cell viability following bioprinting, bioprinted constructs were stained using LIVE/DEAD kits (Life Technologies). Briefly, the constructs were incubated for 1 hour with concentrations of 2 μM calcein-AM and 4 μM ethidium homodimer-1 in a 1:1 mixture of PBS and HCM. After staining, constructs which were fixed with 4% paraformaldehyde for 60 minutes and washed with PBS. The constructs were then imaged using a Leica TCS LSI macro-confocal microscope. Z-stacks of 150 μm were taken of each construct, from which maximum projections were obtained. For use in subsequent experiments, only batches organoids with viabilities of over 90% were employed (not shown).

Integration with Microfluidic Microreactor Devices.

Microfluidic devices were fabricated by assembly of PDMS components formed by conventional soft lithography and replica molding.³¹ The micro-bioreactors consist of PDMS (polydimethylsiloxane) blocks to guide fluid flow, that are held tightly from the top and bottom by PMMA clamps. The fabrication process started by machining two PMMA (polymethyl methacrylate) clamps that will secure the PDMS structures inside the bioreactor and will facilitate the addition of other structures. The PMMA layers were machined using laser cutting (3-mm) PMMA (8560K239, McMaster). The bottom PMMA clamp had eight 2-mm holes on the edge of a 15×10 mm rectangle. The top part consisted of the same aligned eight holes (for screws clamping) and two 3.5 mm holes, with their centers aligned to the inlet/outlet posts of the micro-bioreactor.

The microfluidic components of the reactor were made using soft lithography of PDMS. To create the molds for the PDMS microfluidics components, PMMA sheets were machined using a laser cutter, or formed using SU-8 photoresist. PDMS prepolymer was prepared by thoroughly mixing the silicone base and the curing agent (10:1 ratio by volume) for 5 min, followed by degassing of the PDMS mixture in a vacuum chamber for 30 minutes. Then, the pre-polymer was poured onto respective positive molds. For the thin lower layer (inlet piece) 2.0 g per 10-cm Petri dish was used, whereas 6.0 g was added for the thicker upper layer (outlet piece). A second degassing procedure was conducted to remove all the bubbles present, followed by curing of the PDMS at 80° C. for at least 90 min. Once cured, the two PDMS layers were cut against a mold. The cell chamber area was cut off from the lower layer, but saved for the plasma-bonding step later. Holes for inlet/outlet connections were cut using 1-mm punch on the upper layer.

Assembly of the system started with the preparation of the bottom layer, which was performed using a standard irreversible air plasma bonding (Plasma Cleaner PDC-32G, Harrick Plasma) of the PDMS bottom layer to the TMSPMA-treated glass slide, such that the chamber faces opposite to the glass slide. Prior to bonding, the glass slide and PDMS layers were be thoroughly cleaned against the scotch tape. Bonded constructs were then kept in the 80° C. oven for overnight.

Next step in the fabrication process of the bioreactor was the insertion of 1 mm connectors into the two punched holes of the top layer. A PMMA structure with corresponding holes was used as a protective layer to contain the PDMS in place near the connection. PDMS pre-polymer was added to completely fill the holes, followed by curing in 80° C. oven for 60 min. After curing, the connectors were carefully removed and PTFE tubing was inserted into the holes and secured by epoxy glue. PDMS pads, which constitute the cushion layer, were prepared by pouring 7.5 grams of degassed PDMS into a 10 cm dish, followed by curing, to generate 1 mm thick PDMS pads. This cushion layer was used between the glass slide and the bottom PMMA cover. For use the layers of the microbioreactor are clamped and screwed to hold them together.

To accept bioprinted organoid constructs, the constructs on coverslips were transferred into the 7 mm by 5 mm organoid chambers micro-bioreactor devices using sterile forceps. Microreactor devices were then sealed and clamped immediately prior to use. Each device was connected by tubing to a microfluidic pump, bubble trap, and media reservoir containing the appropriate media type depending on the subsequent experimental conditions (HCM, CMM, or a 50:50 common media). Flow was initiated at 10 uL/min and maintained to fill the system.

Liver Construct Synthetic Functionality, Response to Acetaminophen Insult, and Intervention with N-Acetyl-L-Cysteine.

To assess the response of the liver organoid system to toxic drug insult, acetaminophen was employed. Liver organoids were cultured in microreactors as described before for 14 days. Media samples were collected on days 3, 6, 10, and 14. After media collection on day 6, 1 set of organoids continued with normal media, 1 set of organoids were treated with 1 mM APAP, and 1 set of organoids were treated with 10 mM APAP. To assess the effectiveness of a countermeasure treatment to be used in the liver organoid system, N-acetyl-L-cysteine was explored as a clinically relevant treatment against APAP-induced toxicity. This final set of organoids was treated with 10 mM APAP and 20 mM N-acetyl-L-cysteine. During media changes, groups receiving the drug treatment received fresh HCM also containing the appropriate drug concentration.

For assessment of liver organoid albumin and urea secretion under baseline conditions as well as during exposure to APAP, collected media aliquots were analyzed using a Human Albumin ELISA assay (Alpha Diagnostic International) and the amount of secreted urea in the collected media was determined using a Urea colorimetric assay (BioAssay Systems). For viability assessment, organoids were removed immediately after the final media collection time point (day 14) for staining by LIVE/DEAD viability/cytotoxicity kits (Life Technologies). Staining consisted of incubation in 2 uM calcein AM (stains live cells green) and 4 uM EthD-1 (stains dead cells red) in a 1:1 PBS:HCM solution. Following staining, organoids were washed in PBS, fixed in 4% PFA, transferred to PBS, and imaged using macro-confocal microscopy (Leica TCS LSI). Additionally, media samples were analyzed for presence of lactate dehydrogenase (LDH), an enzyme that is released from cells after toxicity causes cell membrane rupture, using a Lactate Dehydrogenase Assay Kit (Abcam), and for α-GST, a hepatocyte-specific enzyme also released from cells after exposure to toxicity, using an α-GST Assay Kit (Oxford Biomedical Research).

Cardiac Construct Baseline Function Monitoring and Beat Rate Response to Drugs.

The onboard camera was designed and fabricated based on a commercial cost effective webcam (Logitech C160) and significantly improved from lens-less versions.^{35,36} The schematics in FIG. 4a show the fabrication procedure of the microscope with parts compiled from a webcam. First the cover of the webcam is disassembled to retrieve the CMOS sensor. The lens of the webcam is then detached from its initial location, flipped, and integrated back to the holder to convert it into a magnifying lens. A base was then constructed for the mini-microscope to fit onto the bottom of the bioreactors. The base consisted of a dual-layer structure of PMMA sheets (⅛″ Thick, 12″×12″, McMaster 8505K11) cut into the dimensions of the bioreactors using a laser cutter (VLS 2.30 Desktop Laser System, Universal Laser Systems). Using 4 sets of screw/bolts, the CMOS module was tightly clamped in between a pair of PMMA structures. Additional 4 sets of screw/bolts were further mounted at the corners of the structures to function as the focus knobs. Only very minor alteration to the bioreactor itself was needed, i.e., 4 extra holes were drilled on the lower PMMA board to fit the imager at the bottom.

During culture of cardiac constructs, videos were captured to analyze cardiac organoid beating rates. Video files were analyzed using custom written MatLab code with a series of MatLab functions. The software created a reference frame, based on the first frame of the video, and compared pixels in each subsequent frame, determining which pixels represented movement over time. The moving pixels in each frame were then used to generate a black and white pixilated representation of beat behavior, allowing visualization of beat propagation, and generation a plot showing the number of moving pixels versus time, allowing determination of beating rates.

To assess cardiac organoid beating rate response to drugs, videos of cardiac organoids were captured under baseline conditions or having been treated with 0.1 mM isoproterenol, 1 μM quinidine, or combinations of epinephrine and propranolol. For the latter two drugs first, epinephrine was administered at the following concentrations and organoid beating rates were determined: 0 μM, 0.1 μM, 1 μM, 10 μM, 50 μM. Next, the response of epinephrine under the influence of propranolol, a beta-blocker that prevents increases in heart rate in vivo, was assessed by initial incubation of cardiac organoids with 0 μM, 0.5 μM, 5 μM, and 20 μM for 15 minutes, after which epinephrine was administered at a concentration of 5 uM, and beating rate was determine visually under the microcope.

Integrated Organoid System and Integrated Response to Drugs.

To evaluate how the combination of both organoid types together impact drug response, epinephrine and propranolol were tested independently and jointly. In the independent scenario, organoid platforms were prepared in two groups: Group 1 consisted of a set of organoids comprised only of cardiac, with “blank” liver modules. Group 2 consisted of both cardiac and liver. However, it should be noted that cardiac and liver constructs were kept separate for the incubation period, while the drug was administered to the liver construct or “blank” liver module, after which the modules were joined for 30 minutes prior to cardiac beating rate assessment. Baseline cardiac organoid beating rates were determined in each group prior to drug administration. Then, the drugs—either 0.1 μM propranolol or 0.5 μM epinephrine—were administered, allowed to incubate for 1 hour, after which the modules were joined, and data was collected.

To test the integrated response of the liver and cardiac system to epinephrine and propranolol combinations, the experimental groups described above were prepared and the same protocol (individual unit incubation prior to joining of modules) was followed. However, the incubation period was lengthened to overnight (18 hours). Both Group 1 and Group 2 were administered 0.1 uM propranolol. After the incubation period, the modules were joined and 0.5 uM epinephrine was be administered to both groups. Additionally, in parallel, a Group 3 condition was employed, which mirrored Group 2, but used a 2D hepatocyte culture (1-2 million cells/well) instead of the liver construct as a 2D comparison.

### Supplementary Materials and Methods

Liver Cell Sources and Culture.

All cells used were commercially sourced, human primary cells. Hepatic stellate cells (HSCs)(ScienCell) were expanded in culture for two passages before cryopreservation for use in organoid formation. During expansion, HSCs were cultured in 90% high glucose DMEM (Gibco) and 10% fetal bovine serum (Atlanta Bio.) on a rat tail collagen I coating (10 ng/cm², Corning) at 37° C. with 5% CO₂. Primary human hepatocytes (Triangle Research Labs) were thawed according to manufacturer instructions using Hepatocyte Thawing Medium (Triangle Research Labs). Kupffer cells were also thawed via manufacturer instructions (Gibco). Two-dimensional hepatocyte sandwich cultures were used as a comparison to the liver organoid. Primary human hepatocytes (Triangle Research Labs) were thawed as mentioned above, then plated on collagen coated (10 ng/cm2, Corning) 6-well culture plates, using Hepatocyte Plating medium (Triangle Research Labs) at a density of ˜150,000 cells/cm². Cells were incubated at 37° C. with 5% CO₂ for 4 hours before adding matrigel as an overlay (BD). Following further incubation for 24 hours, fresh HCM medium (Lonza) was added.

Cardiac Cell Sources and Culture.

Induced pluripotent stem cell-derived cardiomyocytes were commercially sourced from Stem Cell Theranostics and organoids were cultured in cardiomyocyte maintenance medium (CMM, Stem Cell Theranostics).

Organoid Viability Assays.

Organoid viability was assessed by ATP production as a measure of metabolic activity as detailed in the following white paper¹. CellTiter-Glo assay (Promega) was used to measure ATP by transferring one organoid/well to a black, opaque 96-well plate (Corning). Blanks were included using HCM medium (Lonza) at 80 μl/well. 80 μl of prepared CellTiter-Glo buffer was added per well and plate was placed on shaker for 5 minutes to lyse cells, then further incubated for 15 minutes protected from light. Plate was read using plate reader (SpectraMax M5, Molecular Devices) with an integration time of 0.5 sec/well. Sample time points were compared via two-sample unequal variance t-test. Live/dead stain was also used to assess viability. Organoids were washed in PBS and then stained with live/dead viability/cytotoxicity kit (Life Technologies): 2 μL/mL ethidium homodimer-1 and 0.5 μL/mL calcein AM (diluted in PBS) for 45 minutes at room temperature, protected from light. Organoids were transferred to a depression glass slide (Erie Scientific) and then imaged using TCS LSI macro confocal microscope with 5× macro objective (Leica).

Organoid Functionality Assays.

Urea and albumin production were measured by collecting supernatant from individual wells 24 hours following medium change. Urea production was measured using a colorimetric assay, Quantichrom Urea Assay Kit, (BioAssay Systems) following manufacturer's instructions. Samples were measured in a 96-well clear assay plate (Corning) using plate reader set to 430 nm (SpectraMax M5, Molecular Devices). Data were analyzed using two-sample unequal variance t-test. Albumin production was measured using Human Albumin ELISA kit (Alpha Diagnostic International) according to manufacturer's instructions. Samples were measured using plate reader set to 450 nm (SpectraMax M5, Molecular Devices) and data were analyzed using two-sample unequal variance t-test.

Individual Organoid Immunohistochemistry.

Preparing organoids for histology. Organoids were collected and fixed in 4% paraformaldehyde for 1 hour at room temperature. Organoids were embedded in Histogel (Richard-Allan Scientific) and then dehydrated with a series of graded ethanol washes before paraffin embedding to be sectioned at 4 m. Sections were stained with hematoxylin and eosin and imaged via light microscopy using a DM4000B microscope (Leica).

Immunohistochemistry.

All washes were performed in TBS buffer and incubation steps at room temperature unless otherwise stated. Sections were deparaffinized and hydrated to water and then a heat induced epitope retrieval step was performed in 0.01M citrate buffer (pH 6.0). Endogenous enzyme activity was blocked using Duel Endogenous Enzyme Block (Dako) incubated for 10 minutes. Slides were blocked in Serum Free Protein Block (Dako) for 15 minutes. Primary antibodies were diluted in Antibody Diluent (Dako) and incubated overnight at 4° C. Antibodies used include: mouse anti-human serum albumin (Abcam, ab10241), rabbit anti-cytokeratin 18 (Abcam, ab52948), rabbit anti-cytochrome P450 reductase (Abcam, ab13513), rabbit anti-GFAP (Abcam, ab7260), rabbit anti-connexin 32 (Invitrogen, 71-0700), and rabbit anti-E-cadherin (Abcam, ab40772), mouse anti-troponin T-C(Santa-Cruz, sc73234). Secondary antibodies were diluted in Antibody Diluent (Dako) and incubated for 1 hour. Secondary antibodies used include: peroxidase AffiniPure donkey anti-rabbit IgG (Jackson ImmunoResearch Labs, 711-035-152), biotin anti-mouse IgG (Vector Labs, BA-2000) and biotin anti-rabbit IgG (Vector Labs, BA-1000). For HRP conjugated antibodies, samples were developed using the NovaRed substrate kit (Vector). For avidin-biotinylated conjugate antibodies, slides were developed using Vectastain Universal ABC-AP kit (Vector) and VectorRed AP substrate (Vector). Slides were stained with hematoxylin and then permanently coverslipped with Mounting Media 24 (Leica). Slides were imaged via light microscopy using DM4000B microscope (Leica).

Whole Mount Organoid Immunofluorescence.

Cardiac organoids were analyzed via whole mount immunofluorescence imaging. All washes were performed with PBS and steps were performed at room temperature unless otherwise stated. Organoids were collected and fixed with 4% paraformaldehyde, incubated for one hour on shaker. Organoids were permeabilized using 0.5% Triton-X 100, incubated for one hour on shaker. Samples were treated with Protein Block (Dako) for one hour. Primary antibodies were diluted in Antibody Diluent (Dako) and incubated overnight at 4° C. Primary antibodies used include: rabbit anti-VEGF (Santa Cruz, sc-152), mouse anti-a-actinin (Santa-Cruz, sc-17829), and mouse anti-MYL7 (Santa-Cruz, sc-365255). Secondary antibodies were diluted in Antibody Diluent (Dako) and incubated overnight at 4° C. Secondary antibodies used were: goat anti-rabbit AF488 (Life Technologies) and goat anti-mouse AF594. Samples were stained with DAPI for 20 minutes on shaker. Samples were transferred to a depression glass slide (Erie Scientific) for imaging using TCS LSI macro confocal with 5× macro objective (Leica).

Mass Spectrometry for Drug Metabolism.

All drug compounds used for this experiment were sourced from Sigma Aldrich. Drug toxicity in the organoids and monolayer cultures was assessed by inducing cytochrome P450 activity using a mixture of rifampicin (25 mM), 3-methylcholanthrene (3.78 μg/mL), and phenobarbital (58.0 μg/mL) in HCM medium (Lonza), inducing the cells for 24 hours. Then diazepam was added (2.5 μg/mL) in HCM medium for 24 hours. Diazepam metabolites temazepam, nordiazepam, and oxazepam were measured in the cell supernatant. Sample volumes were measured with 4-OH coumarin added as an internal standard to a final concentration of 500 pg/μl, and 25 μl injected onto a Phenomenex Hypersil 3 μm C18-BD 150 mm length×2 mm I.D. column (P/N 00F-4018-B0), maintained at 50° C. and eluted at a flow rate of 0.2 ml/min. The LC gradient was as follows: 95% A at 0 min., to 30% A from 0-6 min., hold at 30% A from 6-20 min., to 95% A from 20-22 min., hold at 95% A from 22-30 min, where solvent A was 95:5 (v/v) H2O:Methanol+0.15% formic acid, and solvent B was methanol+0.15% formic acid. The system used was a Thermo-Scientific Quantum Discovery Max triple quadrupole mass spectrometer run in positive ion and multiple reaction monitoring modes, automated by a Spark Holland LC, and a Reliance auto-sampler and conditioned stacker maintained at 4° C. The spray voltage was 3500V, the capillary temperature was 250° C., the scan time was 0.1 seconds, the Q1 and Q3 peak widths were both 0.70, and the Q2 collision gas pressure was 0.8 mtorr.

Troglitazone Toxicity.

Troglitazone (Sigma-Aldrich) stock solutions were suspended in DMSO (Sigma-Aldrich) and then diluted in HCM medium at concentrations of 0 μM, 1 μM, 1.67 μM, 2 μM, 2.33 μM, 2.67 μM, and 3 μM. A DMSO toxicity control was made with 1% DMSO in HCM medium and all treatment stocks contained <1% DMSO. Organoids were treated with troglitazone for 48 hours before collecting samples. Organoid viability was measured using the CellTiter-Glo assay (Promega) as recorded as previously described. Accumulation of phospholipids within the organoids was imaged using the HCS LipidTox Phospholipidosis Detection Stain (Invitrogen). LipidTox reagent was added to medium at the same time as the troglitazone at a ratio of 1:500. Following 48 hour drug treatment, organoids were fixed in 4% paraformaldehyde (Sigma Aldrich), washed in PBS, and then transferred to a depression glass slide (Erie Scientific) for imaging using TCS LSI macro confocal with 5× macro objective (Leica).

Phenotype and Long-Term Viability Characterization of Microreactor-Cultured Liver Constructs.

For phenotype characterization via immunostaining, organoids were maintained in culture for up to 28 days, during which several analyses were performed at various time points. Spent media was replaced with fresh HCM on day 3, day 6, 10, 14, 17, 21, 24, and 28. After 8 days, organoid constructs were fixed in 4% PFA and rinsed in PBS, after which constructs were maintained in PBS at 4° C. until processing for histological analysis (described below). For albumin and urea secretion analysis organoids were maintained in culture for 14 days, during which media was collected and replaced with fresh HCM on days 3, 7, 10, and 14. For viability assessment, organoids were maintained in culture for up to 28 days. Subsets of organoids were removed from microreactor culture on day 1, day 14, and day 28 for staining by LIVE/DEAD viability/cytotoxicity kits (Life Technologies), after which they were fixed in 4% PFA, transferred to PBS, and imaged using macro-confocal microscopy (Leica TCS LSI)

Fixed liver constructs were carefully removed from plastic coverslips, paraffin processed (graded ethanol washes, xylene, and paraffin), and prepared for tissue sectioning. Tissue sections (5 μm) on glass microscope slides were prepared using a microtome. For IHC, all incubations were carried out at room temperature unless otherwise stated. Slides were warmed at 60° C. for 1 hr to increase bonding to the slides. Antigen retrieval was performed on all slides and achieved with incubation in Proteinase K (Dako, Carpinteria, Calif.) for 5 min. Sections were permeabilized by incubation in 0.05% Triton-X for 5 min. Non-specific antibody binding was blocked by incubation in Protein Block Solution (Abcam) for 15 min. Sections were incubated for 60 min in a humidified chamber with the primary albumin (raised in mouse, cat. # A6684, Sigma), CYP3A4 (raised in rabbit, cat. # NBP1-95969, Novus Biologicals, Littleton, Colo.), Ost-Alpha (raised in rabbit, cat. # sc-100078, Santa Cruz, Dallas, Tex.), dipeptidyl peptidase-4 (raised in rabbit, cat. # ab28340), E-cadherin (raised in mouse, cat. #610181, BD Biosciences, San Jose, Calif.), ZO-1 (raised in rabbit, cat. #61-7300, Invitrogen), or ß-catenin (raised in rabbit, cat. #71-2700, Invitrogen), all at 1:200 dilutions in antibody diluent (Abcam).

Following primary incubation, slides were washed 3 times in PBS for 5 min. Samples were then incubated for 1 hr with anti-rabbit or anti-mouse Alexa Fluor 488 secondary antibodies (Invitrogen) or an anti-mouse Dylight 594 secondary antibody as appropriate in antibody diluent (1:200 dilution). Cells were counterstained with DAPI for 5 minutes, and washed 3 times with 1×PBS prior to fluorescent imaging. Negative controls were performed in parallel with the primary antibody incubations and included incubation with blocking solution in place of the primary antibody. No immunoreactivity was observed in the negative control sections. Samples were imaged with fluorescence at 488 nm, 594 nm, and 380 nm with a Leica DM 4000B upright microscope.

Onboard sensor implementation. Physical sensors. The operation of the oxygen sensor was based on quenching of an exogenous photoluminescent dye under the presence of oxygen (Papkovsky, D. B. & Dmitriev, R. I. Biological detection by optical oxygen sensing. Chem Soc Rev 42, 8700-8732, doi:10.1039/c3cs60131e (2013)), and is described in more detail in Zhang, Y. S., et al. (Zhang, Y. S. et al. A cost-effective fluorescence mini-microscope with adjustable magnifications for biomedical applications. Lab Chip 15: 3661-9 (2015)). The sensor consisted of an UV light source, an excitation filter (460 nm, Thorlabs) and an emission filter (630 nm, Thorlabs), and an oxygen-sensitive dye deposited on the glass slide. The glass slide was cleaned thoroughly with ethanol, and plasma treated for 90 seconds. Then, a piece of Scotch tape was placed on the slide and a square opening in the tape was cut using a laser cutter. Tris(4,7-diphenyl-1,10-phenanthroline) ruthenium(II) dichloride (AlfaAesar) in ethanol was dispensed on the glass slide, and evaporated in the dark, leaving a layer of the dye. The tape was removed, leaving the dried layer of dye. In order to protect the dye from washing away by fluid flow, a thin layer of PDMS was coated over the dye on the slide by spin coating at 500 rpms for 10 seconds and subsequently to 6,000 rpm for 60 seconds. Then, the slide was cured at 80° C. for 30 minutes. The glass slide was then bonded to a PDMS channel, using plasma treatment, with space to accommodate pH, oxygen and temperature sensors. The channel had one inlet and one outlet connecting the sensing module to the main fluid circuit. To minimize the required volume, the three sensors share a single channel.

The operation of the pH sensor was based on UV light absorption at the phenol red-containing media at different pH levels flowing through the sensor channel as described in Zhang, above. Specifically, sensing focuses on the absorption spectra of phenol red-containing Dulbecco's Modified Eagle's Medium (DMEM) at pH values between 6-8. There are two major absorption peaks at approximately 420 nm and 560 nm. The distinction between the peaks at different pH values was more prominent at 560 nm compared to 420 nm. Taking advantage of the different adsorption levels of phenol red containing DMEM at pH values, the optical sensor was developed. The sensor consisted of a white light LED as a light source (Radioshack), a photo-diode (FDS100, Thorlabs) and a long-pass filter (495 nm, Thorlabs) that were assembled and connected to a PDMS fluid channel. The long-pass filter was utilized to obtain a linear calibration curve on the voltage reading (mV) at different pH values. The high pass filter with a cut-off wavelength of 495 nm was mounted in front of the photodiode to remove signals with wavelengths below 495 nm. When illuminated with a broadband LED, the photodiode at the bottom of the bioreactor detected the absorption of light within the phenol red added to the culture media, which correlated linearly with pH values in the medium.

The temperature sensor was comprised of a flexible thermocouple microprobe (IT-18, Physitemp Instrument Inc, USA) and a thermocouple measurement interface device (NI USB-TC01, from National Instrument). A sterilized thermocouple microprobe was placed in direct contact with the culture media to measure the temperature. The resolution of temperature sensor was 0.1° C. In order to integrate the temperature sensor in the channel, a hole with a diameter of 1 mm was punched into the PDMS channel before its bonding to the glass slide. Two holes with the same diameter were punched as the inlet and outlet ports. Tubing was used to connect the sensing module to the breadboard and the temperature microprobe was secured in place using a fast drying epoxy.

Data acquisition from the sensors was carried out and controlled by a data acquisition card from National Instrument (NI) and a custom-coded LabVIEW program. In addition, the program controlled the illumination duration for the while LED and the UV LED through electrical relays. Outputs from the photodiodes of the pH and oxygen sensors were collected using the data acquisition card. The temperature sensor had a built-in program for data acquisition that enabled its integration with the in-house developed LabVIEW program.

Electrochemical Sensors.

In order to detect biomarkers without a specific electrochemical reaction such as a mediator, electrochemical impedance spectroscopy (EIS) was employed as the measurement technique. EIS is an electrochemical technique that allows the investigation of the electrical properties of the electrode surfaces and binding kinetics of molecules between the electrolyte and the electrode surface. To capture biomarkers, antibodies or aptamers are used as the bioreceptors' affinity element to capture biomarkers, due to their selectivity and sensitivity against different antigens. A 3-electrode cell is used to perform electro analytical chemistry: the auxiliary (counter) electrode and reference electrode, along with the working electrode, provide the circuit over which current is either applied or measured. Potassium ferricyanide (K3[Fe(CN)6]) electrolyte is added to the test solution to ensure sufficient conductivity. The combination of the electrolyte and specific working electrode material (Au) determines the range of the applied potential. In brief, the attachment of antibodies to an electrode surface introduces a charge transfer resistance to the system.

Electrochemical analysis by cyclic voltammetry (CV) and square wave voltammetry (SWV) EIS were performed using a CHI 660E electrochemical workstation (CH Instruments). For the EIS technique, the initial potential was set to 0.05V and the range of frequencies was scanned from 0.1 Hz to 10 kHz. In SWV the potential was increased from −0.5 V to 0.5 V with steps of 25 mV of amplitude, and an increment between two consecutive steps of 4 mV. The frequency was set at 30.1 Hz and the sensitivity scale was 0.0001 A/V. In the case of CV, the potential range was scanned from −0.5 V to 0.5 V with a scan rate of 0.05 V/s. The entire detection took 6 segments (3 cycles), and the sensitivity was set at 0.00001 A/V. All measurements were carried out in 5 mM K3 [Fe(CN)6] redox probe system. Electrochemical detection was conducted using commercially available screen-printed gold electrodes (Dropsens). The Dropsens electrodes were composed of Au as the auxiliary and the working electrodes, and silver electrode as the reference electrode.

The size of ceramic substrate is 33 mmÅ˜10 mmÅ˜0.5 mm (length Å˜ width Å˜ height). The area of the working electrode is 4π mm².

The surfaces of the electrodes were functionalized by immobilizing streptavidin (SPV) on the working electrode through covalent bonding between the self-assembled monolayer (SAM) (carboxylic groups) and SPV (amine groups) by EDC/NHS (N-[3-dimethylaminopropyl]-N′-ethylcarbodiimide hydrochloride/N-hydroxysuccinimide). The SAM solution was prepared with mercaptoundercanoic acid (10 mM) in ethanol. The Au electrode was incubated within SAM solution for 1 hour at room temperature and then the electrode was washed with ethanol. To create covalent linkers on the SAM layer, a 50 mM EDC/NHS mixture in citric acid (pH 4.5) was added on SAM functionalized electrodes for 15 min. No washing step was required at this point, and the surface was simply dried to remove the excess EDC/NHS. Then the electrode was incubated in SPV (10 μg/ml) for 1 hr. After washing, biotin functionalized antibodies (10 μg/ml) were immobilized on the SPV functionalized electrodes during 1 hr incubation. In case of aptamers, they were immobilized on the electrodes after the EDC/NHS step without using SPV. The bioreceptor functionalized electrodes were incubated in DMEM based cell culture media with 10% FBS and 1% PS which was used as the blocking solution.

Statistical Analysis.

All quantitative results are presented as mean±standard deviation (SD). Experiments were performed in triplicate or greater. Values were compared using Student's t-test (2-tailed) with two sample unequal variance, and p<0.05 or less was considered statistically significant.

### Example 2

### 3d Bioprinting of Rat Heart Tissue

In this study, 3D bioprinting was applied to fabricate functional and contractile cardiac tissue constructs. Rat neonatal heart tissues were obtained to isolate cardiomyocytes, and the cells were suspended in a fibrin-based hydrogel bioink. Cell-laden hydrogel was printed through a 300-micron nozzle by pneumatic pressure. The bioprinted cardiac tissue constructs showed spontaneous contraction after 3 days post-printing and demonstrated synchronized contraction after 14 days in culture, indicating of cardiac tissue development and maturation. Cardiac tissue formation was confirmed by immunostaining with antibodies specific to α-actinin and connexin 43, which showed aligned, dense matured cardiomyocytes. The bioprinted cardiac tissue constructs also showed physiological responses (beating frequency and contraction forces) to known cardiac drugs (epinephrine and carbachol). Moreover, tissue development of the printed cardiac tissue could accelerate by Notch signal blockade. These results demonstrated the feasibility of printing functional cardiac tissues that can be used in model pharmacological applications.

### REFERENCES

1 Jamieson, L. E., Harrison, D. J. & Campbell, C. J. Chemical analysis of multicellular tumour spheroids. The Analyst 140, 3910-3920, doi:10.1039/c5an00524h (2015).
2 Sung, J. H. et al. Using physiologically-based pharmacokinetic-guided “body-on-a-chip” systems to predict mammalian response to drug and chemical exposure. Experimental biology and medicine 239, 1225-1239, doi:10.1177/1535370214529397 (2014).
3 Bhushan, A. et al. Towards a three-dimensional microfluidic liver platform for predicting drug efficacy and toxicity in humans. Stem Cell Res Ther 4 Suppl 1, S16, doi:10.1186/scrt377 (2013).
4 Klinghoffer, R. A. et al. A technology platform to assess multiple cancer agents simultaneously within a patient's tumor. Science translational medicine 7, 284ra258, doi: 10.1126/scitranslmed.aaa7489 (2015).
Kunz-Schughart, L. A., Freyer, J. P., Hofstaedter, F. & Ebner, R. The use of 3-D cultures for high-throughput screening: the multicellular spheroid model. J Biomol Screen 9, 273-285, doi: 10.1177/1087057104265040 (2004).
6 Pasirayi, G. et al. Low cost microfluidic cell culture array using normally closed valves for cytotoxicity assay. Talanta 129, 491-498, doi:10.1016/j.talanta.2014.06.020 (2014).
7 Ho, W. J. et al. Incorporation of multicellular spheroids into 3-D polymeric scaffolds provides an improved tumor model for screening anticancer drugs. Cancer science 101, 2637-2643, doi:10.1111/j.1349-7006.2010.01723.x (2010).
8 Drewitz, M. et al. Towards automated production and drug sensitivity testing using scaffold-free spherical tumor microtissues. Biotechnol J 6, 1488-1496, doi:10.1002/biot.201100290 (2011).
9 Polini, A. et al. Organs-on-a-chip: a new tool for drug discovery. Expert opinion on drug discovery 9, 335-352, doi:10.1517/17460441.2014.886562 (2014).
Benam, K. H. et al. Engineered in vitro disease models. Annual review of pathology 10, 195-262, doi:10.1146/annurev-pathol-012414-040418 (2015).
11 Jouin, D. et al. Cryopreserved human hepatocytes in suspension are a convenient high throughput tool for the prediction of metabolic clearance. Eur J Pharm Biopharm 63, 347-355, doi:10.1016/j.ejpb.2006.01.014 (2006).
12 Kaneko, T., Kojima, K. & Yasuda, K. An on-chip cardiomyocyte cell network assay for stable drug screening regarding community effect of cell network size. The Analyst 132, 892-898, doi:10.1039/b704961g (2007).
13 Peters, M. F., Scott, C. W., Ochalski, R. & Dragan, Y. P. Evaluation of cellular impedance measures of cardiomyocyte cultures for drug screening applications. Assay Drug Dev Technol 10, 525-532, doi:10.1089/adt.2011.442 (2012).
14 Lin, C., Ballinger, K. R. & Khetani, S. R. The application of engineered liver tissues for novel drug discovery. Expert opinion on drug discovery 10, 519-540, doi:10.1517/17460441.2015.1032241 (2015).
Skardal, A. et al. Tissue specific synthetic ECM hydrogels for 3-D in vitro maintenance of hepatocyte function. Biomaterials 33, 4565-4575, doi:S0142-9612(12)00321-3 [pii] 10.1016/j.biomaterials.2012.03.034 (2012).
16 Murphy, S. V. & Atala, A. 3D bioprinting of tissues and organs. Nat Biotechnol 32, 773-785, doi:10.1038/nbt.2958 (2014).
17 Skardal, A. & Atala, A. Biomaterials for integration with 3-d bioprinting. Ann BiomedEng 43, 730-746, doi:10.1007/s10439-014-1207-1 (2015).
18 Skardal, A., Zhang, J. & Prestwich, G. D. Bioprinting vessel-like constructs using hyaluronan hydrogels crosslinked with tetrahedral polyethylene glycol tetracrylates. Biomaterials 31, 6173-6181, doi:S0142-9612(10)00561-2 [pii]10.1016/j.biomaterials.2010.04.045 (2010).
19 Smith, A. S. et al. Microphysiological systems and low-cost microfluidic platform with analytics. Stem Cell Res Ther 4 Suppl 1, S9, doi: 10.1186/scrt370 (2013).
Esch, M. B. et al. How multi-organ microdevices can help foster drug development. Adv Drug Deliv Rev 69-70, 158-169, doi:10.1016/j.addr.2013.12.003 (2014).
21 Asthana, A. & Kisaalita, W. S. Microtissue size and hypoxia in HTS with 3D cultures. Drug Discov Today 17, 810-817, doi:10.1016/j.drudis.2012.03.004 (2012).
22 Kelm, J. M. et al. Design of artificial myocardial microtissues. Tissue Eng 10, 201-214, doi:10.1089/107632704322791853 (2004).
23 Messner, S., Agarkova, I., Moritz, W. & Kelm, J. M. Multi-cell type human liver microtissues for hepatotoxicity testing. Arch Toxicol 87, 209-213, doi:10.1007/s00204-012-0968-2 (2013).
24 Ambrosino, G. et al. Isolated hepatocytes versus hepatocyte spheroids: in vitro culture of rat hepatocytes. Cell Transplant 14, 397-401 (2005).
Tostoes, R. M. et al. Human liver cell spheroids in extended perfusion bioreactor culture for repeated-dose drug testing. Hepatology 55, 1227-1236, doi: 10.1002/hep.24760 (2012).
26 Wu, F. J., Friend, J. R., Remmel, R. P., Cerra, F. B. & Hu, W. S. Enhanced cytochrome P450 IA1 activity of self-assembled rat hepatocyte spheroids. Cell Transplant 8, 233-246 (1999).
27 Yoo, J. J. et al. Delivery System. United States of America patent US 2011/0172611 A1 (2011).
28 Skardal, A. et al. A hydrogel bioink toolkit for mimicking native tissue biochemical and mechanical properties in bioprinted tissue constructs. Acta Biomater, doi:10.1016/j.actbio.2015.07.030 (2015).
29 Murphy, S. V., Skardal, A. & Atala, A. Evaluation of hydrogels for bioprinting applications. J Biomed Mater Res A 101, 272-284, doi:10.1002/jbm.a.34326 (2013).
Skardal, A. et al. Photocrosslinkable hyaluronan-gelatin hydrogels for two-step bioprinting. Tissue Eng Part A 16, 2675-2685, doi:10.1089/ten.TEA.2009.0798 (2010).
31 Xia, Y. & Whitesides, G. M. Soft Lithography. Annu Rev Mater Scie 28, 153-184 (1998).
32 Zhang, Y. S. et al. An efficient bubble trapping system with continuous bubble removal capability for microfluidics applications. Biomed Microdevices In press (2015).
33 Zhang, Y. S. et al. A cost-effective fluorescence mini-microscope with adjustable magnifications for biomedical applications. Lab Chip 15 (2015).
34 Han, K. N., Li, C. A. & Seong, G. H. Microfluidic chips for immunoassays. Annual review of analytical chemistry 6, 119-141, doi:10.1146/annurev-anchem-062012-092616 (2013).
35 Kim, S. B. et al. A cell-based biosensor for real-time detection of cardiotoxicity using lensfree imaging. Lab Chip 11, 1801-1807, doi:10.1039/c11c20098d (2011).
36 Kim, S. B. et al. A mini-microscope for in situ monitoring of cells. Lab Chip 12, 3976-3982, doi:10.1039/c21c40345e (2012).

The foregoing is illustrative of the present invention, and is not to be construed as limiting thereof. The invention is defined by the following claims, with equivalents of the claims to be included therein.

1. A method of making a cardiac construct, comprising:
depositing a mixture comprising live mammalian cardiac cells, fibrinogen, gelatin, and water on a support to form an intermediate cardiac construct;
optionally co-depositing a structural support material with said mixture in a configuration that supports said intermediate construct; and then
contacting thrombin to said construct in an amount effective to cross-link said fibrinogen and produce a cardiac construct comprised of live cardiac cells that together spontaneously beat in a fibrin hydrogel.

2. The method of claim 1, wherein said cardiac cells are in the form of organoids produced by hanging drop culture of cardiomyocytes and/or 3d bioprinting thereof.

3. The method of claim 1, wherein said cardiac construct exhibits spontaneous beating that is increased in frequency by the administration of isoproterenol in an effective amount and decreased in frequency by the administration of quinidine in an effective amount.

4. The method of claim 1, wherein cardiac cells of the cardiac construct express VEGF, actinin, and/or cardiac troponin-T.

5. A cardiac construct produced by the process of claim 1.

6. An apparatus, comprising:
a first chamber having an inlet and an outlet; and
a cardiac construct in said primary chamber, said cardiac construct comprising a cross-linked fibrin hydrogel, and cardiac cells that spontaneously beat together in said hydrogel.

7. The apparatus of claim 6, wherein cardiac cells of the cardiac construct express VEGF, actinin, and/or cardiac troponin-T.

8. The apparatus of claim 6, further comprising:
a cardiac monitor operatively associated with said cardiac construct.

9. The apparatus of claim 6, further comprising:
at least one secondary chamber in fluid communication with said primary chamber; and
a live mammalian liver tissue construct in said secondary chamber.

10. The apparatus of claim 6, further comprising:
at least one additional secondary chamber in fluid in communication with said primary and/or secondary chambers; and
at least one additional live tissue construct in each said additional secondary chamber.

11. The apparatus of claim 6, further comprising:
a growth media in said primary chamber, each said secondary chamber, and said conduits therebetween.

12. The apparatus of claim 6, further comprising an optically transparent window in said primary and/or secondary chambers.

13. The apparatus of claim 6, further comprising a fluid inlet connected to said primary chamber and a fluid outlet connected to each said secondary chamber.

14. The apparatus of claim 6, wherein said secondary chambers are connected to one another in series, in parallel, or in combinations thereof.

15. The apparatus of claim 6, further comprising a pump operatively associated with said primary chamber for circulating said growth media from said primary chamber to said secondary chamber.

16. The apparatus of claim 6, further comprising a growth media reservoir and/or bubble trap operatively associated with said primary chamber.

17. The apparatus of claim 6, further comprising a return conduit operatively associated with said primary and secondary chambers (and said pump, and reservoir and/or bubble trap when present) for returning growth media circulated through said secondary chambers to said primary chamber.

18. The apparatus of claim 6, packaged in a container with a transient protective support media in said primary and secondary chambers in gelled form, and optionally together with a cooling element in said container.

19. A method of screening at least one test compound for physiological activity, comprising the steps of:
providing an apparatus of claim 6;
optionally circulating a growth medium from said first chamber to said second chamber;
administering at least one test compound to said constructs; and
determining a change in beat frequency of said cardiac construct as compared to that observed when said test compound is not administered.

20. The method of claim 19, wherein said at least one test compound comprises at least two distinct test compounds that are administered concurrently with one another.

21. The method of claim 19, wherein said determining step is carried out a plurality of times sequentially spaced from one another.
